(19) United States
(12) Patent Application Publication
(10) Pub. No.: US 2025/0281227 A1
(43) Pub. Date: Sep. 11, 2025

(54) USER INTERFACE FOR SURGICAL INSTRUMENT WITH COMBINATION ENERGY MODALITY END-EFFECTOR

(71) Applicant: Cilag GmbH International, Zug (CH)

(72) Inventors: Jeffrey D. Messerly, Cincinnati, OH (US); Craig N. Faller, Batavia, OH (US); Justin S. Cramer, Loveland, OH (US); Steven M. Boronyak, Cincinnati, OH (US); Ellen E. Burkart, Liberty Township, OH (US); Monica L.Z. Rivard, Cincinnati, OH (US); Gregory J. Bakos, Mason, OH (US)

(21) Appl. No.: 19/095,778

(22) Filed: Mar. 31, 2025

Related U.S. Application Data
(63) Continuation of application No. 16/887,499, filed on May 29, 2020, now Pat. No. 12,262,937.
(60) Provisional application No. 62/955,294, filed on Dec. 30, 2019.

(51) Int. Cl. A61B 18/14; A61B 18/00; A61B 18/12

(52) U.S. Cl. CPC A61B 18/14; A61B 18/1206; A61B 2018/0019; A61B 2018/00607; A61B 2018/0063; A61B 2018/0072; A61B 2018/00732; A61B 2018/00767; A61B 2018/00916; A61B 2018/00922; A61B 2018/00958; A61B 2018/00994; A61B 2018/1253; A61B 2018/126; A61B 2018/1412

(57) ABSTRACT

Disclosed is a surgical instrument having a housing, an end-effector, and a user interface. The end-effector includes a clamp arm and an ultrasonic blade configured to couple to an ultrasonic transducer and to a pole of an electrical generator. The clamp arm includes a clamp jaw pivotally movable about a pivot point, an electrode defining a surface configured to contact tissue and apply electrical energy to the tissue in contact therewith and configured to couple to an opposite pole of the electrical generator. The user interface includes a first activation button switch to activate a first energy source and a second button switch to select an energy mode for the activation button switch.

### CROSS-REFERENCE TO RELATED APPLICATIONS

This application is a continuation application claiming priority under 35 U.S.C. § 120 to U.S. patent application Ser. No. 16/887,499, titled USER INTERFACE FOR SURGICAL INSTRUMENT WITH COMBINATION ENERGY MODALITY END-EFFECTOR, filed May 29, 2020, now U.S. Patent Application Publication No. 2021/0196345, which claims priority under 35 U.S.C. § 119(e) to U.S. Provisional Patent Application Ser. No. 62/955,294, titled USER INTERFACE FOR SURGICAL INSTRUMENT WITH COMBINATION ENERGY MODALITY END-EFFECTOR, filed Dec. 30, 2019, the entire disclosures of which are herein incorporated by reference in their entirety.

### TECHNICAL FIELD

The present disclosure generally relates to user interfaces for surgical instruments with end-effectors adapted and configured to operate with multiple energy modalities to enable tissue sealing and cutting employing simultaneously, independently, or sequentially applied energy modalities. More particularly, the present disclosure relates to user interfaces for surgical instruments with end-effectors adapted and configured to operate with surgical instruments that employ combined ultrasonic and electrosurgical systems, such as monopolar or bipolar radio frequency (RF), to enable tissue sealing and cutting employing simultaneously, independently, or sequentially applied ultrasonic and electrosurgical energy modalities. The energy modalities may be applied based on tissue parameters or other algorithms. The end-effectors may be adapted and configured to couple to hand held or robotic surgical systems.

### BACKGROUND

Ultrasonic surgical instruments employing ultrasonic energy modalities are finding increasingly widespread applications in surgical procedures by virtue of the unique performance characteristics of such instruments. Depending upon specific instrument configurations and operational parameters, ultrasonic surgical instruments can provide substantially simultaneous cutting of tissue and hemostasis by coagulation, desirably minimizing patient trauma. The cutting action is typically realized by an end-effector, or blade tip, at the distal end of the instrument, which transmits ultrasonic energy to tissue brought into contact with the end-effector. An ultrasonic end-effector may comprise an ultrasonic blade, a clamp arm, and a pad, among other components. Ultrasonic instruments of this nature can be configured for open surgical use, minimally invasive procedures such as laparoscopic procedures, for example, or non-invasive procedures such as endoscopic surgical procedures, for example. The instruments may be controlled by a surgeon using a hand held instrument or a robot.

Some surgical instruments utilize ultrasonic energy for both precise cutting and controlled coagulation. Ultrasonic energy cuts and coagulates by vibrating a blade in contact with tissue. Vibrating at high frequencies (e.g., 55,500 times per second), the ultrasonic blade denatures protein in the tissue to form a sticky coagulum. Pressure exerted on tissue with the blade surface collapses blood vessels and allows the coagulum to form a hemostatic seal. The precision of cutting and coagulation is controlled by the surgeon's technique and adjusting the power level, blade edge, tissue traction, and blade pressure.

Electrosurgical devices for applying electrical energy modalities to tissue to treat, seal, cut, and/or destroy tissue also are finding increasingly widespread applications in surgical procedures. An electrosurgical device typically includes an instrument having a distally-mounted end-effector comprising one or more than one electrode. The end-effector can be positioned against the tissue such that electrical current is introduced into the tissue.

Electrosurgical devices can be configured for bipolar or monopolar operation. During bipolar operation, current is introduced though a first electrode (e.g., active electrode) into the tissue and returned from the tissue through a second electrode (e.g., return electrode). During monopolar operation, current is introduced into the tissue by an active electrode of the end-effector and returned through a return electrode such as a grounding pad, for example, separately coupled to the body of a patient. Heat generated by the current flowing through the tissue may form hemostatic seals within the tissue and/or between tissues and thus may be particularly useful for sealing blood vessels, for example. The end-effector of an electrosurgical device also may include a cutting member that is movable relative to the tissue and the electrodes to transect the tissue. Electrosurgical end-effectors may be adapted and configured to couple to hand held instruments as well as robotic instruments.

Electrical energy applied by an electrosurgical device can be transmitted to the instrument by a generator in communication with the hand piece. The electrical energy may be in the form of radio frequency (“RF”) energy. RF energy is a form of electrical energy that may be in the frequency range of 200 kilohertz (kHz) to 1 megahertz (MHz). In application, an electrosurgical device can transmit low frequency RF energy through tissue, which causes ionic agitation, or friction, in effect resistive heating, thereby increasing the temperature of the tissue. Because a sharp boundary is created between the affected tissue and the surrounding tissue, surgeons can operate with a high level of precision and control, without sacrificing un-targeted adjacent tissue. The low operating temperatures of RF energy is useful for removing, shrinking, or sculpting soft tissue while simultaneously sealing blood vessels. RF energy works particularly well on connective tissue, which is primarily comprised of collagen and shrinks when contacted by heat.

The RF energy may be in a frequency range described in EN 60601-2-2:2009+A11:2011, Definition 201.3.218—HIGH FREQUENCY. For example, the frequency in monopolar RF applications may be typically restricted to less than 5 MHz. However, in bipolar RF energy applications, the frequency can be almost anything. Frequencies above 200 kHz can be typically used for monopolar applications in order to avoid the unwanted stimulation of nerves and muscles that would result from the use of low frequency current. Lower frequencies may be used for bipolar applications if the risk analysis shows the possibility of neuromuscular stimulation has been mitigated to an acceptable level. Normally, frequencies above 5 MHz are not used in order to minimize the problems associated with high frequency leakage currents. Higher frequencies may, however, be used in the case of bipolar applications. It is generally recognized that 10 mA is the lower threshold of thermal effects on tissue.

A challenge of utilizing these medical devices is the inability to control and customize single or multiple energy modalities depending on the type of tissue being treated. It would be desirable to provide end-effectors that overcome some of the deficiencies of current instruments and improve the quality of tissue treatment, sealing, or cutting or combinations thereof. The combination energy modality end-effectors described herein overcome those deficiencies and improve the quality of tissue treatment, sealing, or cutting or combinations thereof.

### SUMMARY

In one aspect, an apparatus is provided for dissecting and coagulating tissue. The apparatus comprises a surgical instrument comprising an end-effector adapted and configured to deliver a plurality of energy modalities to tissue at a distal end thereof. The energy modalities may be applied simultaneously, independently, or sequentially. A generator is electrically coupled to the surgical instrument and is configured to supply a plurality of energy modalities to the end-effector. In one aspect, the generator is configured to supply electrosurgical energy (e.g., monopolar or bipolar radio frequency (RF) energy) and ultrasonic energy to the end-effector to allow the end-effector to interact with the tissue. The energy modalities may be supplied to the end-effector by a single generator or multiple generators.

In one aspect, the present disclosure provides a surgical instrument. The surgical instrument comprises a housing, an end-effector, and a user interface. The end-effector comprises a clamp arm and an ultrasonic blade configured to couple to an ultrasonic transducer and to a pole of an electrical generator. The clamp arm comprises a clamp jaw pivotally movable about a pivot point and an electrode defining a surface configured to contact tissue and apply electrical energy to the tissue in contact therewith. The electrode is configured to couple to an opposite pole of the electrical generator. The user interface comprises a first activation button switch to activate a first energy source and a second button switch to select an energy mode for the activation button switch.

In addition to the foregoing, various other method and/or system and/or program product aspects are set forth and described in the teachings such as text (e.g., claims and/or detailed description) and/or drawings of the present disclosure.

The foregoing is a summary and thus may contain simplifications, generalizations, inclusions, and/or omissions of detail; consequently, those skilled in the art will appreciate that the summary is illustrative only and is NOT intended to be in any way limiting. Other aspects, features, and advantages of the devices and/or processes and/or other subject matter described herein will become apparent in the teachings set forth herein.

In one or more various aspects, related systems include but are not limited to circuitry and/or programming for effecting herein-referenced method aspects; the circuitry and/or programming can be virtually any combination of hardware, software, and/or firmware configured to affect the herein-referenced method aspects depending upon the design choices of the system designer. In addition to the foregoing, various other method and/or system aspects are set forth and described in the teachings such as text (e.g., claims and/or detailed description) and/or drawings of the present disclosure.

Further, it is understood that any one or more of the following-described forms, expressions of forms, examples, can be combined with any one or more of the other following-described forms, expressions of forms, and examples.

The foregoing summary is illustrative only and is not intended to be in anyway limiting. In addition to the illustrative aspects, embodiments, and features described above, further aspects, embodiments, and features will become apparent by reference to the drawings and the following detailed description.

### FIGURES

The novel features of the described forms are set forth with particularity in the appended claims. The described forms, however, both as to organization and methods of operation, may be best understood by reference to the following description, taken in conjunction with the accompanying drawings in which:

FIG. 1 illustrates a surgical device comprising a mode selection button switch on the device, according to at least one aspect of the present disclosure.

FIGS. 2A-2C illustrate three options for selecting the various operating modes of the surgical device 100, according to at least one aspect of the present disclosure, where:

FIG. 2A shows a first mode selection option;

FIG. 2B sows a second mode selection option; and

FIG. 2C shows a third mode selection option.

FIG. 3 illustrates a surgical device comprising a mode selection button switch on the back of the device, according to at least one aspect of the present disclosure.

FIG. 4A shows a first mode selection option where as the mode button switch is pressed to toggle through various modes a visual indicator indicates the selected mode.

FIG. 4B shows a second mode selection option where as the mode button switch is pressed to toggle through various modes a visual indicator indicates the selected mode.

FIG. 4C shows a third mode selection option where as the mode button switch is pressed to toggle through various modes a visual indicator indicates the selected mode.

FIG. 4D shows a fourth mode selection option where as a visual indicator in the form of labeled button switches is pressed to select a mode, a visual indicator indicates the selected mode.

FIG. 5 illustrates a surgical device comprising a trigger activation mechanism, according to at least one aspect of the present disclosure.

FIGS. 6A-6B illustrates a framework for energy mode selection and switching for a surgical device, according to at least one aspect of the present disclosure.

FIG. 7 illustrates various shaft light indicators with optional digital overlay disposed on a distal end of the shaft near the end-effector to indicate the operating mode the surgical device, according to at least one aspect of the present disclosure.

FIG. 8 illustrates a surgical device comprising a physical shaft light indicator (e.g., a visual indicator) disposed on a distal end of a shaft coupled to an end-effector, according to at least one aspect of the present disclosure.

FIG. 9 illustrates a surgical device comprising a physical shaft light indicator (e.g., a visual indicator) disposed on a distal end of a shaft coupled to an end-effector, according to at least one aspect of the present disclosure.

FIG. 10 is a cross sectional schematic layout view of four LED physical shaft light indicators (e.g., visual indicators) disposed at the distal end of a shaft of a surgical device, according to at least one aspect of the present disclosure.

FIG. 11 is a flexible circuit with four embedded LED physical shaft light indicators (e.g., visual indicators) attached thereto, according to at least one aspect of the present disclosure.

FIG. 12 is a cross sectional schematic layout view of two LEDs physical shaft light indicators with a light pipe (e.g., a visual indicator) or tube with a co-molded light pipe disposed at the distal end of a shaft of a surgical device, according to at least one aspect of the present disclosure.

FIG. 13 is a light pipe or tube with co-molded light pipe (e.g., a visual indicator) for use with the surgical device shown in FIG. 12, according to at least one aspect of the present disclosure.

FIGS. 14-15 illustrate a surgical device comprising a single physical shaft light indicator (e.g., visual indicators) comprising multi-color LEDS disposed on a distal end of a shaft coupled to an end-effector, according to at least one aspect of the present disclosure.

FIGS. 16-17 illustrate a surgical device comprising physical shaft light indicators (e.g., visual indicators) comprising multiple staggered single color LEDS disposed on a distal end of a shaft coupled to an end-effector, according to at least one aspect of the present disclosure.

FIG. 18 is diagram of a pre-defined algorithm, according to at least one aspect of the present disclosure.

FIG. 19 is a graphical illustration of impedance, power, and cumulative energy for combined (simultaneous) application of ultrasonic and RF energy versus normalized time, according to at least one aspect of the present disclosure.

FIG. 20 is a graphical illustration of impedance, power, and cumulative energy for sequential application of ultrasonic and RF energy versus normalized time, according to at least one aspect of the present disclosure.

FIG. 21 is diagram of a user-defined algorithm, according to at least one aspect of the present disclosure.

FIG. 22 is a surgical system comprising a surgical hub paired with a visualization system, a robotic system, and an intelligent instrument, in accordance with at least one aspect of the present disclosure.

FIG. 23 illustrates an example of a generator, in accordance with at least one aspect of the present disclosure.

FIG. 24 is a diagram of various modules and other components that are combinable to customize modular energy systems, in accordance with at least one aspect of the present disclosure.

FIG. 25A is a first illustrative modular energy system configuration including a header module and a display screen that renders a graphical user interface (GUI) for relaying information regarding modules connected to the header module, in accordance with at least one aspect of the present disclosure.

FIG. 25B is the modular energy system shown in FIG. 25A mounted to a cart, in accordance with at least one aspect of the present disclosure.

FIG. 26 depicts a perspective view of an exemplary surgical system having a generator and a surgical instrument operable to treat tissue with ultrasonic energy and bipolar RF energy, in accordance with at least one aspect of the present disclosure.

FIG. 27 depicts a top perspective view of an end effector of the surgical instrument of FIG. 26, having a clamp arm that provides a first electrode and an ultrasonic blade that provides a second electrode, in accordance with at least one aspect of the present disclosure.

FIG. 28 depicts a bottom perspective view of the end effector of FIG. 27, in accordance with at least one aspect of the present disclosure.

FIG. 29 depicts a partially exploded perspective view of the surgical instrument of FIG. 26, in accordance with at least one aspect of the present disclosure.

FIG. 30 depicts an enlarged exploded perspective view of a distal portion of the shaft assembly and the end effector of the surgical instrument of FIG. 26, in accordance with at least one aspect of the present disclosure.

### DESCRIPTION

Applicant of the present application owns the following U.S. Provisional Patent Applications, filed on Dec. 30, 2019, the disclosure of each of which is herein incorporated by reference in its respective entirety:
U.S. Provisional Patent Application Ser. No. 62/955,292, entitled COMBINATION ENERGY MODALITY END-EFFECTOR;
U.S. Provisional Patent Application Ser. No. 62/955,299, entitled ELECTROSURGICAL INSTRUMENTS FOR COMBINATION ENERGY DELIVERY; and
U.S. Provisional Patent Application Ser. No. 62/955,306, entitled SURGICAL INSTRUMENTS.

Applicant of the present application owns the following U.S. Patent Applications that were filed on May 29, 2020, and which are each herein incorporated by reference in their respective entireties:
U.S. patent application Ser. No. 16/887,493, entitled METHOD OF OPERATING A COMBINATION ULTRASONIC/BIPOLAR RF SURGICAL DEVICE WITH A COMBINATION ENERGY MODALITY END-EFFECTOR, now U.S. Patent Application Publication No. 2021/0196334;
U.S. patent application Ser. No. 16/887,506, entitled DEFLECTABLE SUPPORT OF RF ENERGY ELECTRODE WITH RESPECT TO OPPOSING ULTRASONIC BLADE, now U.S. Pat. No. 11,786,291;
U.S. patent application Ser. No. 16/887,515, entitled NON-BIASED DEFLECTABLE ELECTRODE TO MINIMIZE CONTACT BETWEEN ULTRASONIC BLADE AND ELECTRODE, now U.S. Pat. No. 12,114,912;
U.S. patent application Ser. No. 16/887,519, entitled DEFLECTABLE ELECTRODE WITH HIGHER DISTAL BIAS RELATIVE TO PROXIMAL BIAS, now U.S. Pat. No. 11,950,797;
U.S. patent application Ser. No. 16/887,532, entitled DEFLECTABLE ELECTRODE WITH VARIABLE COMPRESSION BIAS ALONG THE LENGTH OF THE DEFLECTABLE ELECTRODE, now U.S. Pat. No. 11,937,863;
U.S. patent application Ser. No. 16/887,554, entitled ASYMMETRIC SEGMENTED ULTRASONIC SUPPORT PAD FOR COOPERATIVE ENGAGEMENT WITH A MOVABLE RF ELECTRODE, now U.S. Pat. No. 11,944,366;
U.S. patent application Ser. No. 16/887,561, entitled VARIATION IN ELECTRODE PARAMETERS AND DEFLECTABLE ELECTRODE TO MODIFY ENERGY DENSITY AND TISSUE INTERACTION, now U.S. Pat. No. 12,053,224;
U.S. patent application Ser. No. 16/887,568, entitled TECHNIQUES FOR DETECTING ULTRASONIC BLADE TO ELECTRODE CONTACT AND REDUCING POWER TO ULTRASONIC BLADE, now U.S. Pat. No. 11,911,063;
U.S. patent application Ser. No. 16/887,576, entitled CLAMP ARM JAW TO MINIMIZE TISSUE STICKING AND IMPROVE TISSUE CONTROL, now U.S. Pat. No. 11,779,387; and
U.S. patent application Ser. No. 16/887,579, entitled PARTIALLY CONDUCTIVE CLAMP ARM PAD TO ENABLE ELECTRODE WEAR THROUGH AND MINIMIZE SHORT CIRCUITING, now U.S. Patent Application Publication No. 2021/0196352.

Applicant of the present application owns the following U.S. Patent Applications that were filed on May 28, 2020, and which are each herein incorporated by reference in their respective entireties:
U.S. patent application Ser. No. 16/885,813, entitled METHOD FOR AN ELECTROSURGICAL PROCEDURE;
U.S. patent application Ser. No. 16/885,820, entitled ARTICULATABLE SURGICAL INSTRUMENT;
U.S. patent application Ser. No. 16/885,823, entitled SURGICAL INSTRUMENT WITH JAW ALIGNMENT FEATURES;
U.S. patent application Ser. No. 16/885,826, entitled SURGICAL INSTRUMENT WITH ROTATABLE AND ARTICULATABLE SURGICAL END EFFECTOR;
U.S. patent application Ser. No. 16/885,838, entitled ELECTROSURGICAL INSTRUMENT WITH ASYNCHRONOUS ENERGIZING ELECTRODES;
U.S. patent application Ser. No. 16/885,851, entitled ELECTROSURGICAL INSTRUMENT WITH ELECTRODES BIASING SUPPORT;
U.S. patent application Ser. No. 16/885,860, entitled ELECTROSURGICAL INSTRUMENT WITH FLEXIBLE WIRING ASSEMBLIES;
U.S. patent application Ser. No. 16/885,866, entitled ELECTROSURGICAL INSTRUMENT WITH VARIABLE CONTROL MECHANISMS;
U.S. patent application Ser. No. 16/885,870, entitled ELECTROSURGICAL SYSTEMS WITH INTEGRATED AND EXTERNAL POWER SOURCES;
U.S. patent application Ser. No. 16/885,873, entitled ELECTROSURGICAL INSTRUMENTS WITH ELECTRODES HAVING ENERGY FOCUSING FEATURES;
U.S. patent application Ser. No. 16/885,879, entitled ELECTROSURGICAL INSTRUMENTS WITH ELECTRODES HAVING VARIABLE ENERGY DENSITIES;
U.S. patent application Ser. No. 16/885,881, entitled ELECTROSURGICAL INSTRUMENT WITH MONOPOLAR AND BIPOLAR ENERGY CAPABILITIES;
U.S. patent application Ser. No. 16/885,888, entitled ELECTROSURGICAL END EFFECTORS WITH THERMALLY INSULATIVE AND THERMALLY CONDUCTIVE PORTIONS;
U.S. patent application Ser. No. 16/885,893, entitled ELECTROSURGICAL INSTRUMENT WITH ELECTRODES OPERABLE IN BIPOLAR AND MONOPOLAR MODES;
U.S. patent application Ser. No. 16/885,900, entitled ELECTROSURGICAL INSTRUMENT FOR DELIVERING BLENDED ENERGY MODALITIES TO TISSUE;
U.S. patent application Ser. No. 16/885,917, entitled CONTROL PROGRAM ADAPTATION BASED ON DEVICE STATUS AND USER INPUT;
U.S. patent application Ser. No. 16/885,923, entitled CONTROL PROGRAM FOR MODULAR COMBINATION ENERGY DEVICE; and
U.S. patent application Ser. No. 16/885,931, entitled SURGICAL SYSTEM COMMUNICATION PATHWAYS.

Before explaining various forms of surgical instruments in detail, it should be noted that the illustrative forms are not limited in application or use to the details of construction and arrangement of parts illustrated in the accompanying drawings and description. The illustrative forms may be implemented or incorporated in other forms, variations and modifications, and may be practiced or carried out in various ways. Further, unless otherwise indicated, the terms and expressions utilized herein have been chosen for the purpose of describing the illustrative forms for the convenience of the reader and are not for the purpose of limitation thereof.

Further, it is understood that any one or more of the following-described forms, expressions of forms, examples, can be combined with any one or more of the other following-described forms, expressions of forms, and examples.

Various forms are directed to user interfaces for surgical instruments with ultrasonic and/or electrosurgical (RF) end-effectors configured for effecting tissue treating, dissecting, cutting, and/or coagulation during surgical procedures. In one form, a user interface is provided for a combined ultrasonic and electrosurgical instrument that may be configured for use in open surgical procedures, but has applications in other types of surgery, such as minimally invasive laparoscopic procedures, for example, non-invasive endoscopic procedures, either in hand held or and robotic-assisted procedures. Versatility is achieved by selective application of multiple energy modalities simultaneously, independently, sequentially, or combinations thereof. For example, versatility may be achieved by selective use of ultrasonic and electrosurgical energy (e.g., monopolar or bipolar RF energy, microwave, IRE) either simultaneously, independently, sequentially, or combinations thereof.

In one aspect, the present disclosure provides a user interface for an apparatus comprising an ultrasonic blade and clamp arm with a deflectable RF electrode such that the ultrasonic blade and deflectable RF electrode cooperate to effect sealing, cutting, and clamping of tissue by cooperation of a clamping mechanism of the apparatus comprising the RF electrode with an associated ultrasonic blade. The clamping mechanism includes a pivotal clamp arm which cooperates with the ultrasonic blade for gripping tissue therebetween. The clamp arm is preferably provided with a clamp tissue pad (also known as “clamp arm pad”) having a plurality of axially spaced gripping teeth, segments, elements, or individual units which cooperate with the ultrasonic blade of the end-effector to achieve the desired sealing and cutting effects on tissue, while facilitating grasping and gripping of tissue during surgical procedures.

In one aspect, the end-effectors described herein comprise an electrode. In other aspects, the end-effectors described herein comprise alternatives to the electrode to provide a compliant coupling of RF energy to tissue, accommodate pad wear/thinning, minimize generation of excess heat (low coefficient of friction, pressure), minimize generation of sparks, minimize interruptions due to electrical shorting, or combinations thereof. The electrode is fixed to the clamp jaw at the proximal end and is free to deflect at the distal end. Accordingly, the electrode may be referred to throughout this disclosure as a cantilever beam electrode or as a deflectable electrode.

In other aspects, the end-effectors described herein comprise a clamp arm mechanism configured to high pressure between a pad and an ultrasonic blade to grasp and seal tissue, maximize probability that the clamp arm electrode contacts tissue in limiting or difficult scenarios, such as, for example, thin tissue, tissue under lateral tension, tissue tenting/vertical tension especially tenting tissue away from clamp arm.

In other aspects, the end-effectors described herein are configured to balance match of surface area/current densities between electrodes, balance and minimize thermal conduction from tissue interface, such as, for example, impacts lesion formation and symmetry, cycle time, residual thermal energy.

In other aspects, the end-effectors described herein are configured to minimize sticking, tissue adherence (minimize anchor points) and may comprise small polyimide pads.

In various aspects, the present disclosure provides a surgical device configured to deliver at least two energy types (e.g., ultrasonic, monopolar, bipolar RF, microwave, or irreversible electroporation [IRE]) to tissue. The surgical device includes a first activation button switch for activating energy, a second button switch for selecting an energy mode for the activation button switch. The second button switch is connected to a circuit that uses at least one input parameter to define the energy mode. The input parameter can be modified remotely through connection to a generator or through a software update.

In one aspect, at least one of the energy modes is a simultaneous blend of RF and ultrasonic energy, and the input parameter represents a duty cycle of the RF and ultrasonic energy.

In one aspect, the second button switch is configurable to select from a list of predefined modes and the number of modes in the list is defined by a second input parameter defined by a user.

In one aspect, the input parameter is either duty cycle, voltage, frequency, pulse width, or current.

In one aspect, the device also includes a visual indicator of the selected energy mode within the portion of device in the surgical field

In one aspect, the second button switch is a separate control from the end effector closure trigger.

In one aspect, the second button switch is configured to be activated second stage of the closure trigger. The first stage of the closure trigger in the closing direction is to actuate the end effector.

In one aspect, at least one of the energy modes is selected from ultrasonic, RF bipolar, RF monopolar, microwave, or IRE.

In one aspect, at least one of the energy modes is selected from ultrasonic, RF bipolar, RF monopolar, microwave, or IRE and is configured to be applied in a predefined duty cycle or pulsed algorithm.

In one aspect, at least one of the energy modes is selected from a sequential application of two or more of the following types of energy: ultrasonic, RF bipolar, RF monopolar, microwave, or IRE.

In one aspect, at least one of the energy modes is a simultaneous blend of two or more of the following types of energy: ultrasonic, RF bipolar, RF monopolar, microwave, and IRE.

In one aspect, at least one of the energy modes is a simultaneous blend of two or more of the following types of energy: ultrasonic, RF bipolar, RF monopolar, microwave, and IRE followed sequentially by one or more of the aforementioned energies.

In one aspect, at least one of the energy modes is one off the following types of energy: Ultrasonic, RF bipolar, RF monopolar, microwave, and IRE followed sequentially by a simultaneous blend of two or more of the aforementioned energies.

In one aspect, at least one of the energy modes is procedure or tissue specific predefined algorithm.

In one aspect, at least one of the energy modes is compiled from learned surgical behaviors or activities.

In one aspect, the input parameter is at least one of: energy type, duty cycle, voltage, frequency, pulse width, current, impedance limit, activation time, or blend of energy.

In one aspect, the second button switch is configurable to select from a list of predefined modes and the number of modes in the list is either predefined or defined by a second input parameter defined by a user.

In one aspect, the aforementioned energy modes are made available to the user through software updates to the generator.

In one aspect, the aforementioned energy modes are made available to the user through software updates to the device.

In one aspect, the preferred selections by the user are made available to multiple generators through either networking, the cloud, or manual transfer.

In one aspect, the device also includes a visual indicator of the selected energy mode within the portion of device in the surgical field.

As used herein a button switch can be a manually, mechanically, or electrically operated electromechanical device with one or more sets of electrical contacts, which are connected to external circuits. Each set of electrical contacts can be in one of two states: either “closed” meaning the contacts are touching and electricity can flow between them, or “open”, meaning the contacts are separated and the switch is electrically non-conducting. The mechanism actuating the transition between these two states (open or closed) can be either an “alternate action” (flip the switch for continuous “on” or “off”) or “momentary” (push for “on” and release for “off”) type.

As surgical devices evolve and become more capable, the number of specialized modes in which they can be operated increases. Adding extra button switches on a device to accommodate these new additional modes would complicate the user interface and make the device more difficult to use. Accordingly, the present disclosure provides techniques for assigning different modes to a single physical button switch, which enables a wider selection of modes without adding complexity to the housing design (e.g., adding more and more button switches). In one aspect the housing is in the form of a handle or pistol grip.

As more specialized modes become available, there is a need to provide multiple modes to a surgeon using the surgical device without creating a complex user interface. Surgeons want to be able to control the mode selection from the sterile field rather than relying on a circulating nurse at the generator. Surgeon want real time feedback so they are confident they know which mode is selected.

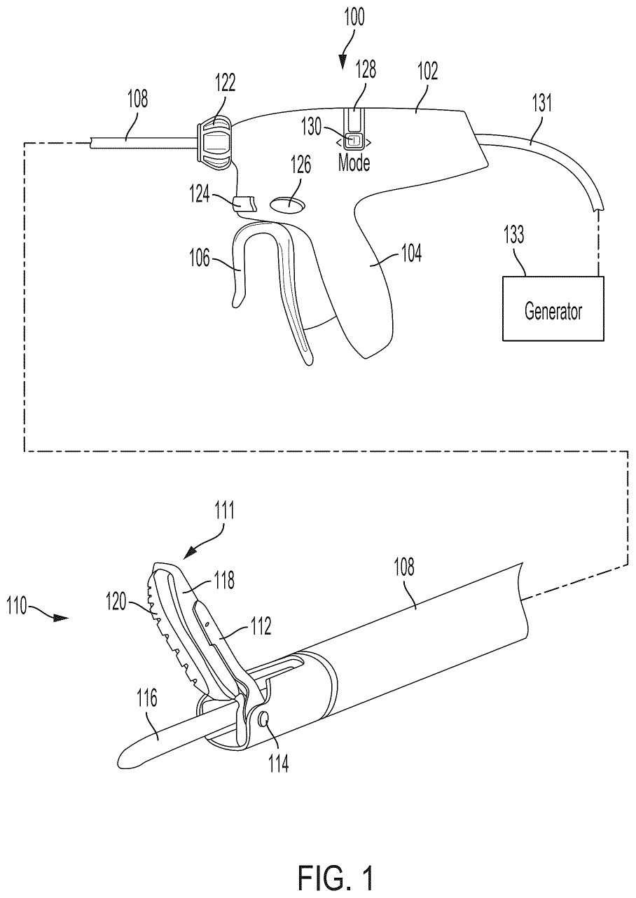

FIG. 1 illustrates a surgical device 100 comprising a mode selection button switch 130 on the device 100, according to at least one aspect of the present disclosure. The surgical device 100 comprises a housing 102 defining a handle 104 in the form of a pistol grip. The housing 102 comprises a trigger 106 which when squeezed is received into the internal space defined by the handle 104. The trigger 106 is used to operate a clamp arm 111 portion of an end-effector 110. A clamp jaw 112 is pivotally movable about pivot point 114. The housing 102 is coupled to the end-effector 110 through a shaft 108, which is rotatable by a knob 122.

The end-effector 110 comprises a clamp arm 111 and an ultrasonic blade 116. The clamp arm 111 comprises a clamp jaw 112, an electrode 118, and a clamp arm pad 120. In one aspect, the clamp arm pad 120 is made of a non-stick lubricious material such as polytetrafluoroethylene (PTFE) or similar synthetic fluoropolymers of tetrafluoroethylene. PTFE is a hydrophobic, non-wetting, high density and resistant to high temperatures, and versatile material and non-stick properties. The clamp arm pad 120 is electrically non-conductive. In contrast, the electrode 118 is made of an electrically conductive material to deliver electrical energy such as monopolar RF, bipolar RF, microwave, or irreversible electroporation (IRE), for example. The electrode 118 may comprises gap setting pads made of a polyimide material, and in one aspect, is made of a durable high-performance polyimide-based plastic known under the tradename VESPEL and manufactured by DuPont or other suitable polyimide, polyimide polymer alloy, or PET (Polyethylene Terephthalate), PEEK (Polyether Ether Ketone), PEKK (Poly Ether Ketone Ketone) polymer alloy, for example. Unless otherwise noted hereinbelow, the clamp arm pads and gap pads described hereinbelow are made of the materials described in this paragraph.

The electrode 118 and the ultrasonic blade 116 are coupled to the generator 133. The generator 133 is configured to drive RF, microwave, or IRE energy to the electrode 118. The generator 133 also is configured to drive an ultrasonic transducer acoustically coupled to the ultrasonic blade 116. In certain implementations, the electrode 118 is one pole of an electrical circuit and the ultrasonic blade 116 is the opposite pole of the electrical circuit. The housing 102 includes a switch 124 to activate the ultrasonic blade 116. The circuit may be contained in the housing 102 or may reside in the generator 133. The surgical device 100 is coupled to the generator 133 via a cable 131. The cable 131 conducts signals for the electrosurgical functions and the ultrasonic transducer.

In various aspects, the surgical device 100 is configured to deliver at least two energy types (e.g., ultrasonic, monopolar RF, bipolar RF, microwave, or irreversible electroporation [IRE]) to tissue located in the end-effector 110 between the clam arm 111 and the ultrasonic blade 116. The housing 102 of the surgical device 100 includes a first activation button switch 126 for activating energy and a second “mode” button switch 130 for selecting an energy mode for the activation button switch. The second button switch 130 is connected to a circuit that uses at least one input parameter to define the energy mode. The input parameter can be modified remotely through connection to a generator or through a software update. The energy mode is displayed on a visual indicator 128, such as display, screen, or similar visual feedback device, to provide feedback to the user concerning the mode or other parameters.

In one aspect, the surgical instrument 100 provides mode switching through the on device directional selector “mode” button switch 130. The user can press the mode button switch 130 to toggle through different modes and the colored light on the visual indicator 128 indicates the selected mode.

According to various aspects of the present disclosure, different modes of operation can be assigned to the surgical device by pressing the “mode” button switch 130, where each time the mode button switch 130 is pressed, or pushed and held, the surgical device 100 toggles through the available modes, which are displayed on the visual indicator 128. Once a mode is selected, the generator 133 will provide the appropriate generator tone and the surgical device 100 will have a lighted indicator on the visual indicator 128 to indicate which mode was selected.

In the example illustrated in FIG. 1, the “mode” selection button switch 130 is placed symmetrically on both sides of the housing 102. This enables both a right and left handed surgeon to select/toggle through modes without using a second hand. In this aspect, the “mode” selection button switch 130 can toggle in many different directions, which enables the surgeon to select from a list of options and navigate more complex selections remotely from the sterile field without having to ask a circulator to make adjustments at the generator 133. The lighted indicator on the visual indicator 128 of the surgical device 100, in addition to generator 133 tones, gives the surgeon feedback on which mode is selected.

In various aspects, the surgical instrument 100 comprises a user interface. The user interface of the surgical instrument 100 comprises, alone or in combination, the first activation button switch 126 to activate a first energy source, the second “mode” button switch 130 to select an energy mode for the first activation button switch 126, and the visual indicator 128. The user interface may further comprises the trigger 106 and the switch 124 to activate a second energy source. The user interface may further comprise the rotatable knob 122 to rotate the shaft 108 and the end-effector 110.

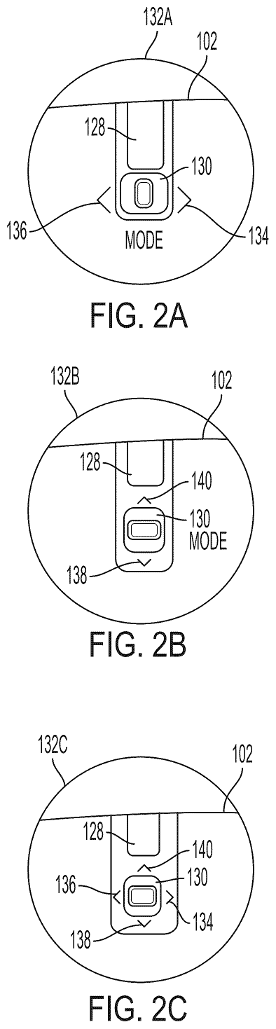

FIGS. 2A-2C illustrate three options for selecting the various operating modes of the surgical device 100, according to at least one aspect of the present disclosure. In addition to the colored light visual indicator 128 on the housing 102 of the surgical device 100, feedback for mode selection is audible and/or visible through the generator 133 interface where the generator 133 announces the selected mode verbally and/or shows a description of the selected mode on a screen of the generator 133.

FIG. 2A shows a first mode selection option 132A where the button switch 130 can be pressed forward 136 or backward 134 to cycle the surgical instrument 100 through the various modes.

FIG. 2B sows a second mode selection option 132B where the button switch 130 is pressed up 140 or down 138 to cycle the surgical instrument 100 through the various modes.

FIG. 2C shows a third mode selection option 132C where the button switch 130 is pressed forward 136, backward 134, up 149, or down 138 to cycle the surgical instrument 100 through the various modes.

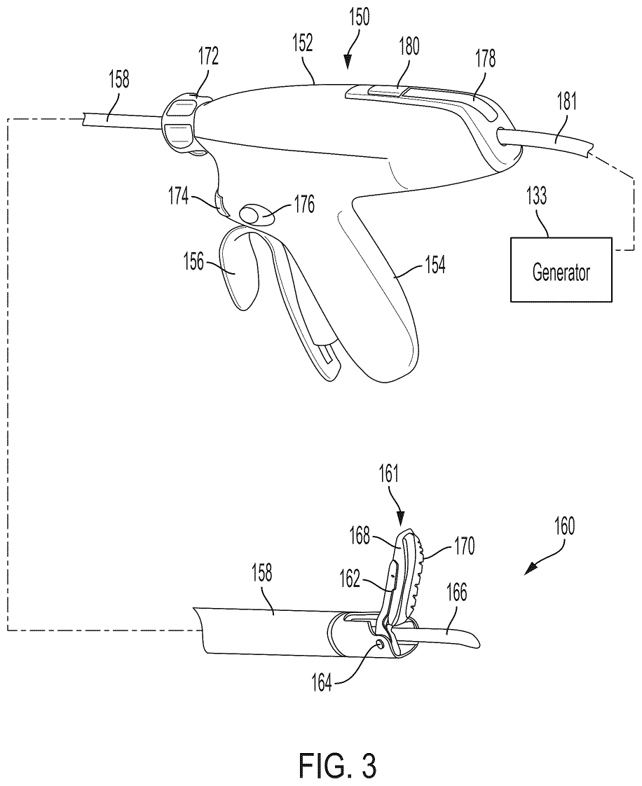

FIG. 3 illustrates a surgical device 150 comprising a mode selection button switch 180 on the back of the device 150, according to at least one aspect of the present disclosure. The surgical device 150 comprises a housing 152 defining a handle 154 in the form of a pistol grip. The housing 152 comprises a trigger 156 which when squeezed is received into the internal space defined by the handle 154. The trigger 156 is used to operate a clamp arm 161 portion of an end-effector 160. A clamp jaw 162 is pivotally movable about pivot point 164. The housing 152 is coupled to the end-effector 160 through a shaft 158, which is rotatable by a knob 172.

The end-effector 160 comprises a clamp arm 161 and an ultrasonic blade 166. The clamp arm 161 comprises a clamp jaw 162, an electrode 168, and a clamp arm pad 170. In one aspect, the clamp arm pad 170 is made of a non-stick lubricious material such as polytetrafluoroethylene (PTFE) or similar synthetic fluoropolymers of tetrafluoroethylene. PTFE is a hydrophobic, non-wetting, high density and resistant to high temperatures, and versatile material and non-stick properties. The clamp arm pad 170 is electrically non-conductive. In contrast, the electrode 168 is made of an electrically conductive material to deliver electrical energy such as monopolar RF, bipolar RF, microwave, or irreversible electroporation (IRE), for example. The electrode 168 may comprises gap setting pads made of a polyimide material, and in one aspect, is made of a durable high-performance polyimide-based plastic known under the tradename VESPEL and manufactured by DuPont or other suitable polyimide, polyimide polymer alloy, or PET (Polyethylene Terephthalate), PEEK (Polyether Ether Ketone), PEKK (Poly Ether Ketone Ketone) polymer alloy, for example. Unless otherwise noted hereinbelow, the clamp arm pads and gap pads described hereinbelow are made of the materials described in this paragraph.

The electrode 168 and the ultrasonic blade 166 are coupled to the generator 133. The generator 133 is configured to drive RF, microwave, or IRE energy to the electrode 168. The generator 133 also is configured to drive an ultrasonic transducer acoustically coupled to the ultrasonic blade 166. In certain implementations, the electrode 168 is one pole of an electrical circuit and the ultrasonic blade 166 is the opposite pole of the electrical circuit. The housing 152 includes a switch 174 to activate the ultrasonic blade 166. The circuit may be contained in the housing 152 or may reside in the generator 133. The surgical device 150 is coupled to the generator 133 via a cable 181. The cable 181 conducts signals for the electrosurgical functions and the ultrasonic transducer.

In various aspects, the surgical device 150 is configured to deliver at least two energy types (e.g., ultrasonic, monopolar RF, bipolar RF, microwave, or irreversible electroporation [IRE]) to tissue located in the end-effector 160 between the clam arm 161 and the ultrasonic blade 166. The housing 152 of the surgical device 150 includes a first activation button switch 176 for activating energy and a second “mode” button switch 180 for selecting an energy mode for the activation button switch. The second button switch 180 is connected to a circuit that uses at least one input parameter to define the energy mode. The input parameter can be modified remotely through connection to a generator 133 or through a software update. The energy mode is displayed on a visual indicator 178 such as a display, screen, or similar visual feedback device.

In one aspect, the surgical instrument 150 provides mode switching through the on device directional selector “mode” button switch 180. The user can press the mode button switch 180 to toggle through different modes and the colored light on the visual indicator 178 indicates the selected mode.

According to various aspects of the present disclosure, different modes of operation can be assigned to the surgical device by pressing the “mode” button switch 180, where each time the mode button switch 180 is pressed, or pushed and held, the surgical device 150 toggles through the available modes, which are displayed on the visual indicator 178. Once a mode is selected, the generator 133 will provide the appropriate generator tone and the surgical device 150 will have a lighted indicator on the visual indicator 178 to indicate which mode was selected.

In the example illustrated in FIG. 3, the “mode” selection button switch 180 is placed on the back of the surgical device 150. The location of the “mode” selection button switch 180 is out of the reach of the surgeon's hand holding the surgical device 150 so a second hand is required to change modes. This is intended to prevent inadvertent activation. In order to change modes, a surgeon must use her second hand to intentionally press the mode button switch 180. The lighted indicator on the visual indicator 178 of the surgical device 150, in addition to generator tones gives the surgeon feedback on which mode is selected.

In various aspects, the surgical instrument 150 comprises a user interface. The user interface of the surgical instrument 150 comprises, alone or in combination, the first activation button switch 176 to activate a first energy source, the second “mode” button switch 180 to select an energy mode for the first activation button switch 176, and the visual indicator 178. The user interface may further comprises the trigger 156 and the switch 174 to activate a second energy source. The user interface may further comprise the rotatable knob 172 to rotate the shaft 158 and the end-effector 160.

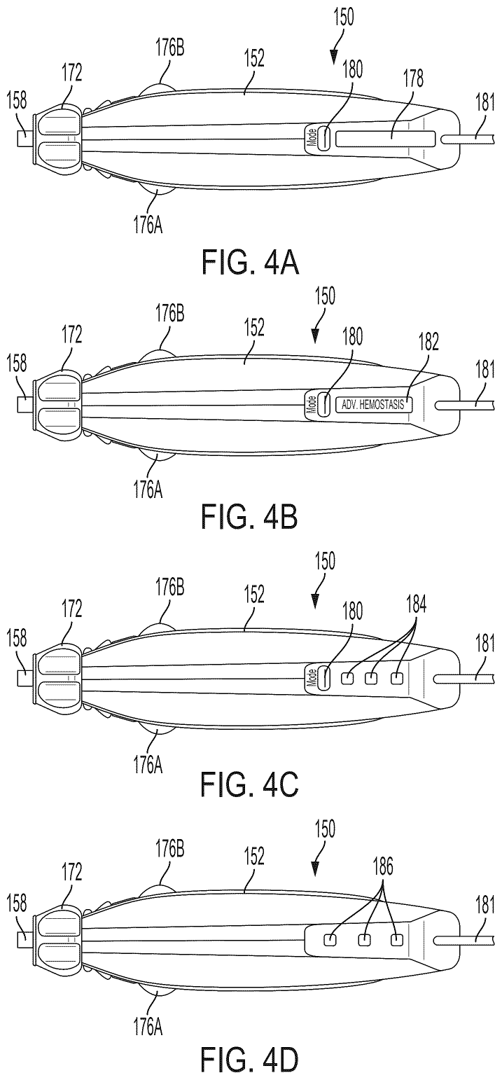

FIG. 4A shows a first mode selection option where as the mode button switch 180 is pressed to toggle through various modes, a visual indicator 178, such as colored light, indicates the selected mode.

FIG. 4B shows a second mode selection option where as the mode button switch 180 is pressed to toggle through various modes, a visual indicator 182 such as display, screen, or similar visual feedback device, indicates the selected mode (e.g., LCD, e-ink).

FIG. 4C shows a third mode selection option where as the mode button switch 180 is pressed to toggle through various modes, a visual indicator 184 such as labelled lights, indicates the selected mode.

FIG. 4D shows a fourth mode selection option where as a visual indicator in the form of labeled button switches 186 is pressed to select a mode, when the selected labeled button switch form of the visual indicator 180 is selected, it is illuminated to indicate mode selected

As more functionality is added to advanced energy surgical devices additional button switches or controls are added to the surgical devices. The additional button switches or controls make these advanced energy surgical devices complicated and difficult to use. Additionally, when using an advanced energy surgical device to control bleeding, difficult to use user interfaces or difficult to access capability will cost critical time and attention during a surgical procedure.

According to the present disclosure, monopolar RF energy or advanced bipolar RF energy is activated by closing the trigger by squeezing the trigger past a first closure click to a second activation click and holding closed until energy delivery is ceased by the power source in the generator. Energy also can be immediately reapplied by slightly releasing and re-squeezing the trigger as many times as desired.

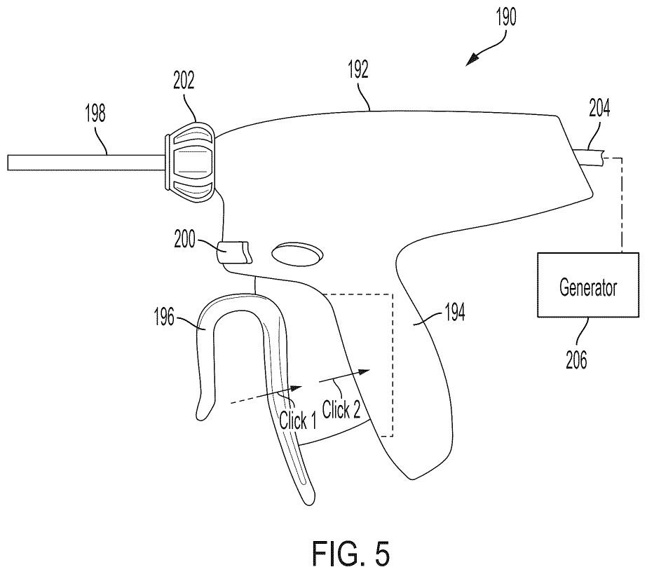

FIG. 5 illustrates a surgical device 190 comprising a trigger 196 activation mechanism, according to at least one aspect of the present disclosure. The surgical device 190 comprises a housing 192 defining a handle 194 in the form of a pistol grip. The housing 192 comprises a trigger 196 which when squeezed is received into the internal space defined by the handle 194. The housing 192 is coupled to an end-effector through a shaft 198, which is rotatable by a knob 202. The surgical device 190 is coupled to a generator 206 via a cable 204. The cable 204 conducts signals for the electrosurgical functions and the ultrasonic transducer.

The trigger 196 is configured to operate a clamp arm portion of an end-effector and to trigger electrosurgical energy, thus eliminating the activation button switch 126, 176 shown in FIGS. 1 and 3. The trigger 196 closes to a first audible and tactile click to close the jaws for grasping tissue and further closes to a second audible and tactile click to activate electrosurgical energy such as monopolar or bipolar RF. Microwave, or IRE energy. The full sequence is completed by activating the front button switch which cuts using ultrasonic energy.

Procedure for operating the surgical device 190: squeeze the trigger 196 to a first audible and tactile click; verify targeted tissue in jaws; activate RF energy by further squeezing the trigger 196 to a second audible and tactile click until end tone is heard; cut by pressing ultrasonic front switch 200 until tissue divides.

Modified procedure for operating the surgical instrument 190 for additional capability: activate RF energy with the trigger 196 and hold while simultaneously activation the front button switch 200 to activate the ultrasonic transducer, which will result in simultaneous application of electrosurgical and ultrasonic energy modalities being delivered to the tissue at the same time.

In an alternative implementation, the front button switch 200 for activating ultrasonic energy may be toggled to different speeds via a mode selector on the surgical device 190 or on the power source generator 206.

In various aspects, the surgical instrument 190 comprises a user interface. The user interface of the surgical instrument 190 comprises, alone or in combination, the trigger 196 configured to close to a first audible and tactile click to close the jaws for grasping tissue and further close to a second audible and tactile click to activate a first energy source such as monopolar or bipolar RF, microwave, IRE, among others, and a display. The user interface may further comprises the switch 200 to activate a second energy source. The user interface may further comprise the rotatable knob 202 to rotate the shaft 198 and the end-effector. The user interface may further comprise a mode switch button to select an energy mode associated with the second audible and tactile click of the trigger 196.

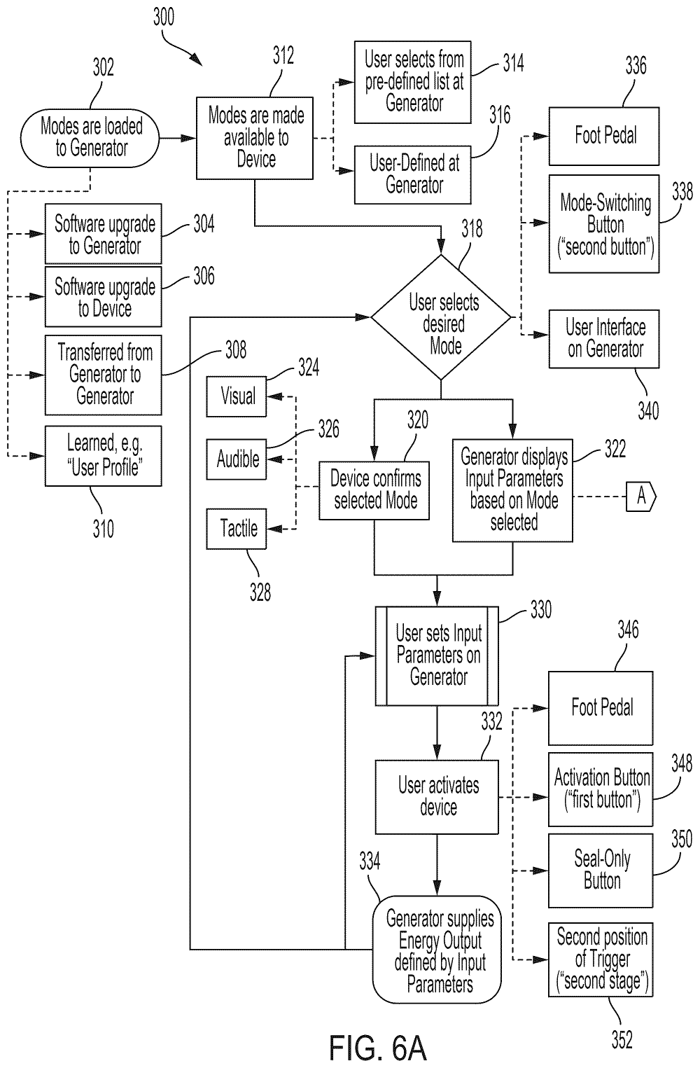

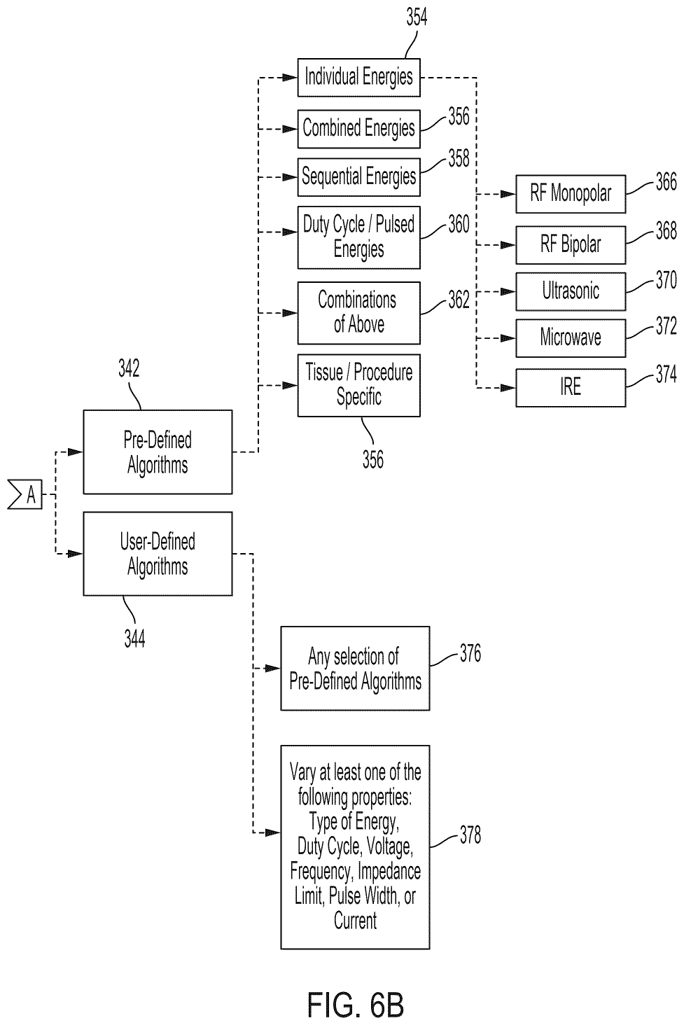

In various aspects, the present disclosure provides the framework for energy mode selection and switching for a surgical instrument such as the surgical instruments 100, 150, 190 descried in FIGS. 1-5. Specifically, the present disclosure provides the framework for energy mode selection and switching for a surgical instrument capable of delivering multiple forms of energy, including both pre-defined and user-defined algorithms of individual energies, combined energies, sequential energies, duty cycle or pulsed energies, tissue-specific or procedure-specific energies, or any combination of above. Specific more in-depth information on each category addressed in the framework 300 depicted in FIGS. 6A-6B can be found throughout this disclosure. The multiple energies include, without limitation, ultrasonic, monopolar RF, bipolar RF, microwave, IRE, applied either independently or in any combination, to seal, cut, feather, blend, etc.

In various aspects, the present disclosure provides mode switching algorithms for a surgical instrument capable of delivering multiple energy options, including both pre-defined algorithms and user-defined algorithms. Pre-defined algorithms include (but not limited to) individual energies, combined energies, sequential energies, duty cycle or pulsed energies, tissue-specific or procedure-specific energies, or any combination of the above.

User-defined algorithms include, without limitation, any selection of pre-defined algorithms or a user-defined sequence of varied electrical outputs.

In various aspects, the present disclosure provides a framework for energy mode selection and switching for a surgical instrument including options for loading different modes to a generator, making modes available to a surgical device, user interface options, and confirmation of selected mode and input parameters, either on the generator or the surgical device, or other user interface option.

FIGS. 6A-6B illustrates a framework 300 for energy mode selection and switching for a surgical device, according to at least one aspect of the present disclosure. The framework 300 is described in the context of the surgical device 100, 150, 190 in FIGS. 1-5 and the generator 133, 206 in FIGS. 1, 3, 5).

The various modes are loaded 302 onto the generator (e.g., generator 133, 206 in FIGS. 1, 3, and 5). This task can be accomplished in multiple different ways, including but not limited to a software upgrade 304 to the generator, a software upgrade 306 to the surgical device (e.g., surgical device 100, 150, 190 in FIGS. 1-5 that is then plugged into the generator with available modes “read” by the generator), a transfer 308 of modes from generator to generator (e.g., via the cloud or other storage solution), or modes could be “learned” 310 by the generator (e.g., via a “User Profile”, or learned surgical behaviors or activities).

The modes are made available 312 to the surgical device. This task can be accomplished in multiple different ways, including but not limited to the user selecting 314 from a pre-defined list of modes at the generator or selecting/defining 316 “User-Defined” modes at the generator.

Whether the user selects 314 from a pre-defined list of modes at the generator or selects/defines 316 “User-Defined” modes at the generator, the user selects 318 the desired mode using several user interface options. The user can interact with, switch between, and select different modes via a mode-switching button switch 338 (e.g., mode button switch 130, 180 in FIGS. 1-4D) on the surgical device via a foot pedal 336, or via the user interface on the generator 340.

Both the generator and the device confirm the mode selected by the user. Example of device confirmation are described herein at sections 278280 and 278282. The surgical device confirms 320 the mode selected by the user. The confirmation 320 of the mode selection can be made by visual 324, audible 326 or tactile 328 feedback. The generator will display 322 the appropriate input parameters based on the mode selected by the user. These input parameters will be defined either by pre-defined algorithms 342 (defined by the manufacturer) or by user-defined algorithms 344 (defined by the user).

Once user selection and confirmation is complete, the user activates 332 the surgical device. The user will be able to activate the selected energy mode (i.e., selected algorithm) in at least one of the following ways from the surgical device: (1) via a foot pedal 346, (2) via the activation button switch 348 (FIGS. 1-4D), (3) via a second activation button switch 350 (i.e., a “Seal Only” button switch), or (4) via a second position of the closure trigger 352 as described in section 279011 (FIG. 5).

Once the device is activated 332, the generator supplies 334 energy output defined by the input parameters. The process continues to monitor the user selection 318 of desired modes and user set 330 input parameters on the generator for any changes and implements the changes as described above.

The pre-defined algorithms 342 may include, for example, individual energies algorithm 354, combined energies algorithm 356, sequential energies algorithm 358, duty cycled/pulsed energies algorithm 360, an algorithm comprising any combination of the above algorithm 354, 356, 358, 360, or tissue/procedure specific algorithm 364. The individual energies algorithm 354 includes, without limitation, RF monopolar 366, RF bipolar 368, ultrasonic 370, microwave 372, and IRE 374 energy modalities.

The user-defined algorithms 344 may include, for example, any selection 376 of pre-defined. Alternatively, the user-defined algorithms 344 may include, for example, vary 378 at least one of the following: type of energy, duty cycle, voltage, frequency, impedance limit, pulse width, or current.

During a surgical procedure, surgeons desire to know what mode the surgical device 100, 150, 190 (FIGS. 1-5) is in at any given point in time without taking their eyes off the surgical laparoscopic field of view. For this reason, a mode indicator would be best placed close to the end-effector such that it is in the line of sight at all times. It is difficult to place LEDs or other physical, dynamic visual indicators on the distal end of the surgical device shaft 108, 158, 198 (FIGS. 1-5) due to manufacturing complexity and limited space in the shaft 108, 158, 198 assembly. Accordingly, it is desirable to provide a surgical device with the functionality to clearly communicate dynamic mode feedback without relying on audible/tone feedback alone.

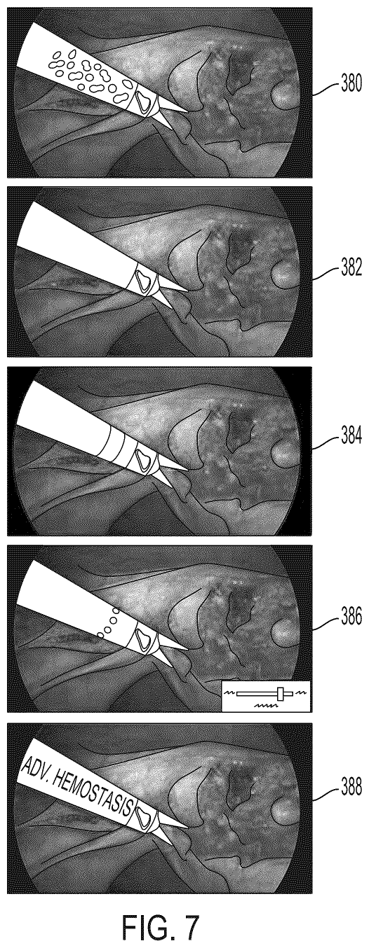

FIG. 7 illustrates various shaft light indicators with optional digital overlay disposed on a distal end of the shaft 108, 158, 198 (FIGS. 1-5) near the end-effector 110, 160 (FIGS. 1-4D) to indicate the operating mode the surgical device 100, 150, 190 (FIGS. 1-5), according to at least one aspect of the present disclosure. A vision system incorporated in a laparoscopic camera may be employed to interpret a static pattern (printed, etched, lasered, etc.) on the distal end of the shaft 108, 158, 198 and digitally overlays a dynamic, visual indicator on the laparoscopic view. Because this is a digital overlay, minimal cost or complexity is added to the surgical device 100, 150, 190. The digital overlay enables many different types of visual indicators and enables them to be customized or dynamically change. Unlike other heads-up display indicators, the present indicator tracks to the pattern on the shaft 108, 158, 198 and enables the mode indicator to follow the surgical device 100, 150, 190 around the surgical field.

The mode switching shaft light indicators with augmented digital overlay are shown in FIG. 7. An un-augmented view 380 shows a physical printed pattern visible on the shaft. A first augmented view 382 shows a physical printed pattern digitally removed in laparoscopic view. A second augmented view 384 shows a mode color indicator digitally overlaid on the shaft. A third augmented view 386 shows mode color indicator adaptability on the shaft. Variables that could be digitally modified include, without limitation, include indicator style/shape, size, color, brightness, or gross positioning (distal/proximal). A fourth augmented view 388 shows a mode described in language on the shaft.

When using a surgical device with mode assignable button switches as disclosed herein in FIGS. 1-6B, it may difficult at times to tell which mode is selected. Although the generator provides audio feedback on mode selected, providing a visual indicator in the line of sight can assist to communicate more clearly when a new mode is assigned.

Surgeons desire to know what mode the surgical device is in at any given point without taking their eyes off the surgical task at hand. For this reason, a visual mode indicator in the line of sight of the surgical task at hand would be best placed close to the end-effector such that the it is in the line of sight of the surgeon at all times.

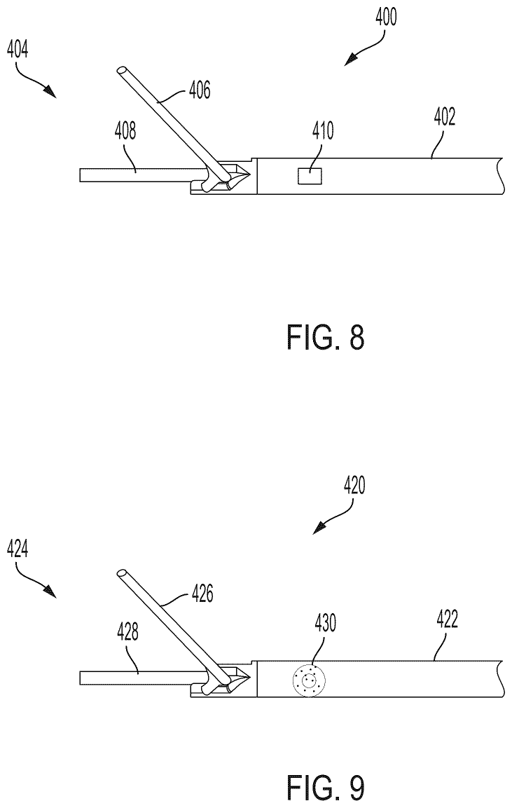

FIGS. 8-17 illustrate various options of physical shaft light indicators placed on the distal end of the shaft 108, 158, 198 (FIGS. 1-5) near the end-effector 110, 160 (FIGS. 1-4D) to indicate the operating mode of the surgical device 100, 150, 190 (FIGS. 1-5), according to at least one aspect of the present disclosure. This placement is visible directly in the laparoscopic camera field of view or, for open procedures, at the surgical site. A lighted indicator could be achieved through a micro-LED placed physically on the distal end of the shaft or remotely through the use of light pipes to transmit colored mode indication to the distal end.

FIG. 8 illustrates a surgical device 400 comprising a physical shaft light indicator 410 (e.g., a visual indicator) disposed on a distal end of a shaft 402 coupled to an end-effector 404, according to at least one aspect of the present disclosure. The end-effector 404 comprises a clamp arm 406 and an ultrasonic blade 408. The physical shaft light indicator 410 disposed on the distal end of the shaft 402 near the end-effector 404 indicates the operating mode of the surgical device 400. In FIG. 8, the surgical device 400 is in default mode and the physical shaft light indicator 410 shows no color. This can be indicated by illuminating a white LED or simply turning off the LED to indicate the default mode of the surgical device 400.

FIG. 9 illustrates a surgical device 420 comprising a physical shaft light indicator 430 (e.g., a visual indicator) disposed on a distal end of a shaft 422 coupled to an end-effector 424, according to at least one aspect of the present disclosure. The end-effector 424 comprises a clamp arm 426 and an ultrasonic blade 428. The physical shaft light indicator 430 disposed on the distal end of the shaft 422 near the end-effector 424 indicates the operating mode of the surgical device 420. In FIG. 8, the surgical device 420 is in a selected mode and the physical shaft light indicator 430 LED is illuminated in color to indicate the selected mode of the surgical device 420.

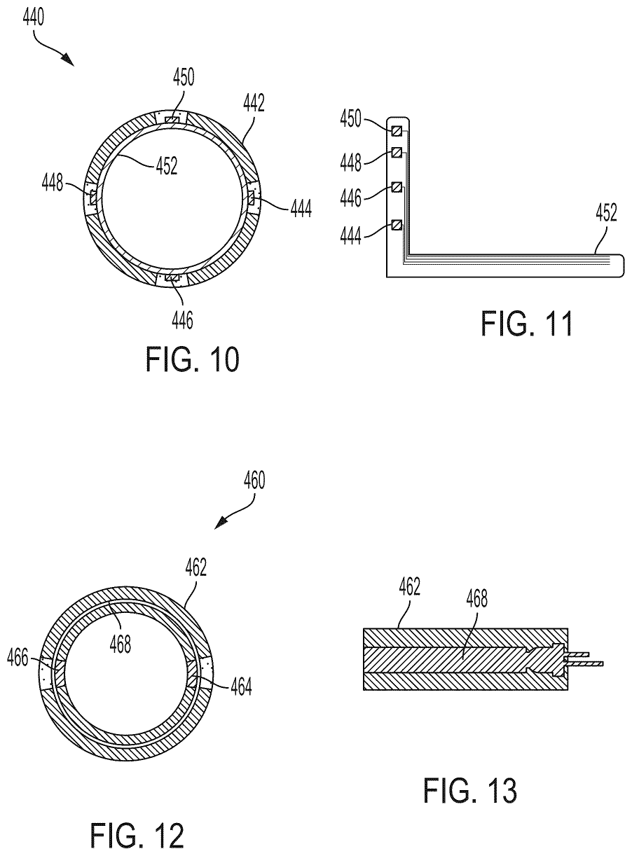

FIG. 10 is a cross sectional schematic layout view of four LED physical shaft light indicators 444, 446, 448, 450 (e.g., visual indicators) disposed at the distal end of a shaft 442 of a surgical device 440, according to at least one aspect of the present disclosure.

FIG. 11 is a flexible circuit 452 with four embedded LED physical shaft light indicators 444, 446, 448, 450 (e.g., visual indicators) attached thereto, according to at least one aspect of the present disclosure. The flexible circuit 452 may be employed in the surgical device 440 shown in FIG. 10.

FIG. 12 is a cross sectional schematic layout view of two LEDs physical shaft light indicators 464, 466 with a light pipe 468 (e.g., a visual indicator) or tube with a co-molded light pipe 468 disposed at the distal end of a shaft 462 of a surgical device 460, according to at least one aspect of the present disclosure.

FIG. 13 is a light pipe 468 or tube with co-molded light pipe 468 (e.g., a visual indicator) for use with the surgical device 460 shown in FIG. 12, according to at least one aspect of the present disclosure.

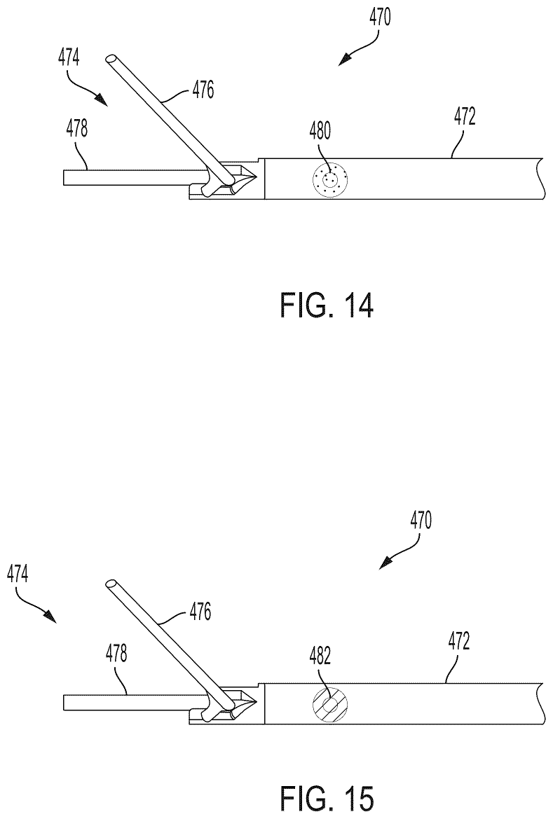

FIGS. 14-15 illustrate a surgical device 470 comprising a single physical shaft light indicator 480, 482 (e.g., visual indicators) comprising multi-color LEDS disposed on a distal end of a shaft 472 coupled to an end-effector 474, according to at least one aspect of the present disclosure. The end-effector 474 comprises a clamp arm 476 and an ultrasonic blade 478. The physical shaft light indicator 480, 482 disposed on the distal end of the shaft 472 indicates the operating mode of the surgical device 470. In FIG. 14, the surgical device 470 is in a first mode and the physical shaft light indicator 480 multi-color LED is illuminated in a first color to indicate the first mode of the surgical device 470. In FIG. 15, the surgical device 470 is in a second mode and the physical shaft light indicator 480 multi-color LED is illuminated in a second color to indicate the second mode of the surgical device 470.

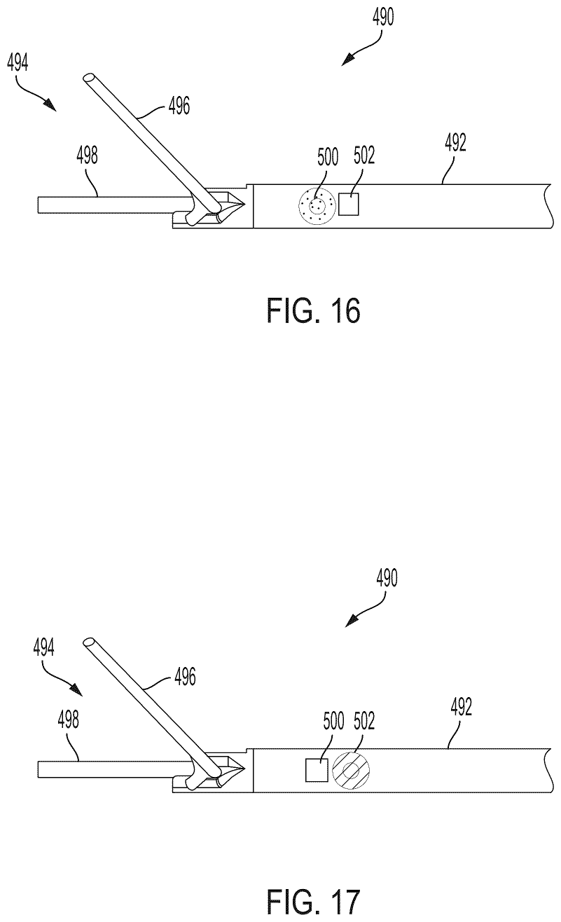

FIGS. 16-17 illustrate a surgical device 490 comprising physical shaft light indicators 500, 502 (e.g., visual indicators) comprising multiple staggered single color LEDS disposed on a distal end of a shaft 492 coupled to an end-effector 494, according to at least one aspect of the present disclosure. The end-effector 494 comprises a clamp arm 496 and an ultrasonic blade 498. The physical shaft light indicators 500, 502 disposed on the distal end of the shaft 492 indicate the operating mode of the surgical device 490. In FIG. 16, the surgical device 490 is in a first mode and the first physical shaft light indicator 500 LED is illuminated in a first color to indicate the first mode of the surgical device 490. In FIG. 17, the surgical device 490 is in a second mode and the second physical shaft light indicator 502 LED is illuminated in a second color to indicate the second mode of the surgical device 490.

The following disclosure provides examples of pre-defined algorithms 342 for mode switching on a combination energy surgical device such as the surgical device 100, 150, 190 described in FIGS. 1-5.

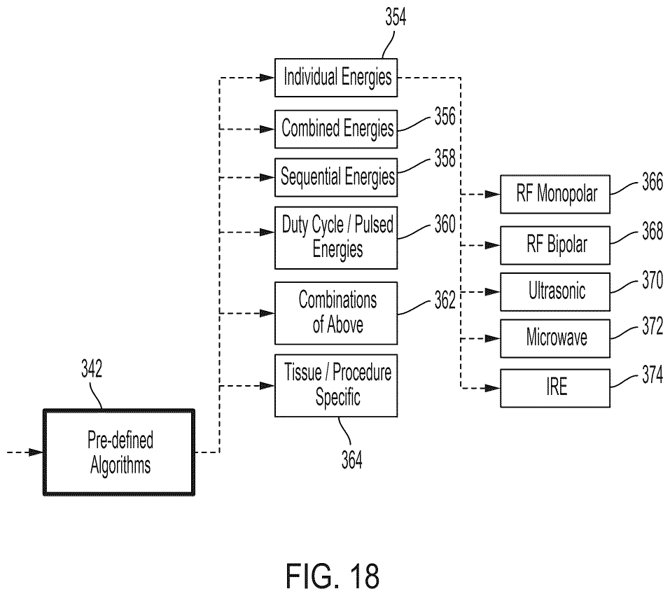

FIG. 18 is diagram of a pre-defined algorithm 342, according to at least one aspect of the present disclosure. With reference to FIGS. 1-6B and as shown in FIG. 18, from a mode switching user interface, the user is able to select from a pre-defined set of algorithms 342. The pre-defined set of algorithms 342 each enable a different type of energy delivery. Energy modalities include, but are not limited to, RF monopolar 366, RF bipolar 368, ultrasonic 370, microwave 372, IRE 374, among others.

A combined energy algorithm 356 enables simultaneous delivery of two or more of the energy modalities 366, 368, 370, 372, 374 listed above. As an example, the combined energy algorithm 356 may deliver power to both RF bipolar electrodes and an ultrasonic transducer that drives an ultrasonic waveguide and an end-effector at the same time to seal and cut a target vessel.

A sequential energy algorithm 358 enables delivery of two or more of the energy modalities listed above in a sequential manner. As an example, the sequential energy algorithm 358 may deliver power to RF bipolar electrodes to seal a target vessel before triggering power to an ultrasonic transducer that drives an ultrasonic waveguide and an end-effector to cut through the sealed target vessel.

A duty cycle/pulsed energy algorithm 360 enables delivery of two or more of the energy modalities 366, 368, 370, 372, 374 listed above in a particular, pre-set order. As an example, the duty cycle/pulsed energy algorithm 360 may cycle quickly between delivering power to RF bipolar electrodes and to an ultrasonic transducer that drives an ultrasonic waveguide and an end-effector to seal but not cut a target vessel, and then pulse power to an ultrasonic transducer to cut through the sealed vessel.

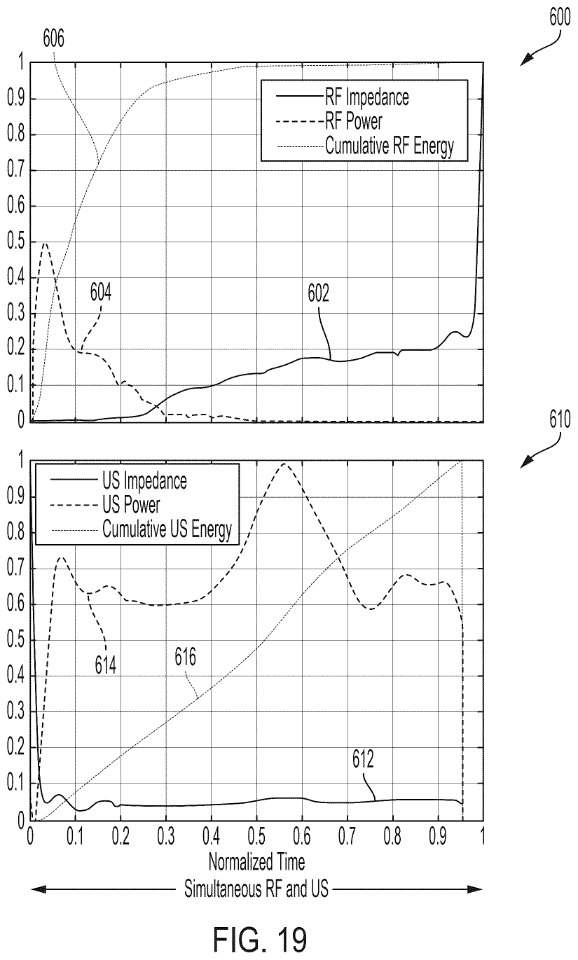

FIG. 19 is a graphical illustration of impedance, power, and cumulative energy for combined (simultaneous) application of ultrasonic and RF energy versus normalized time, according to at least one aspect of the present disclosure. The top graph 600 illustrates the behavior of RF impedance 602, RF power 604, and cumulative RF energy 606 over normalized time during the simultaneous application of RF and ultrasonic energy. The bottom graph 610 illustrates the behavior of ultrasonic (US) impedance 612, US power 614, and cumulative US energy 616 over normalized time during the simultaneous application of RF and ultrasonic energy. With reference to the top and bottom graphs 600, 610, while the cumulative US energy 616 rises in an approximately steady linear manner, the cumulative RF energy 606 rises non-linearly in a rapid exponential manner and then plateaus asymptotically to a maximum cumulative RF energy 606. The illustrated simultaneous application of cumulative RF energy 606 and cumulative US energy achieves sealing and cutting functions.

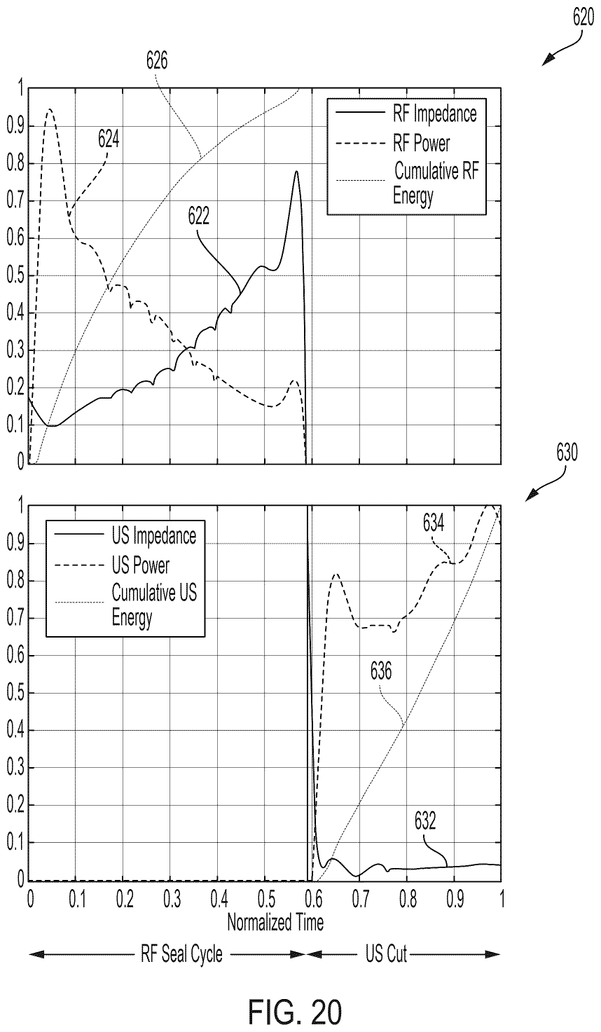

FIG. 20 is a graphical illustration of impedance, power, and cumulative energy for sequential application of ultrasonic and RF energy versus normalized time, according to at least one aspect of the present disclosure. The top graph 620 illustrates the initial application of RF energy with no ultrasonic (US) energy applied. The top graph 620 illustrates the behavior of RF impedance 622, RF power 624, and cumulative RF energy 626 over normalized time during the sole application of RF energy. The bottom graph 630 illustrates the subsequent application of US energy with no RF energy applied. The bottom graph 630 illustrates the behavior of US impedance 632, US power 634, and cumulative US energy 636 over normalized time during the sole application of US energy. With reference to the top and bottom graphs 620, 630, while the cumulative RF energy 626 rises exponentially until it reaches a maximum, the cumulative US energy 632 is zero. When the cumulative RF energy 626 reaches a maximum indicating that a seal has been made, the RF energy is shut off the cumulative RF energy 626 drops to zero. The US energy is then turned on for cutting the sealed tissue. The cumulative US energy 632 rises in steady linear manner sharply to a maximum until the cut is completed.

The following disclosure provides examples of user-defined algorithms 344 for mode switching on a combination energy surgical device such as the surgical device 100, 150, 190 described in FIGS. 1-5.

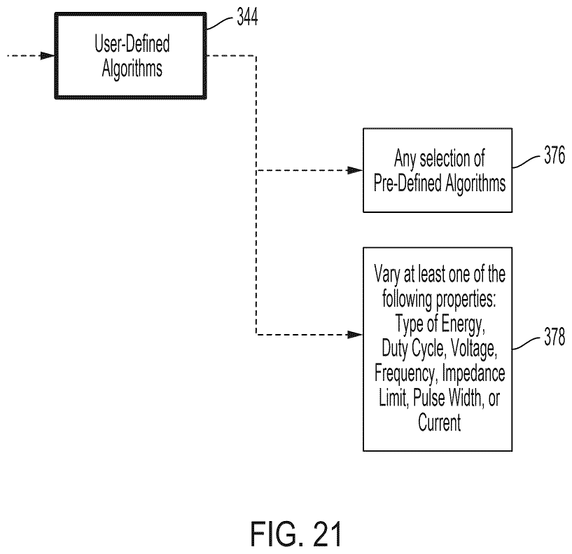

FIG. 21 is diagram of a user-defined algorithm 344, according to at least one aspect of the present disclosure. With reference to FIGS. 1-6B, 18, and as shown in FIG. 21, from a mode switching user Interface, the user is able to either select from a pre-defined set of algorithms 376 or to create/select a user-defined algorithm 378, which could vary at least one of the following properties: type of energy, duty cycle, voltage, frequency, impedance limit, pulse width, current. The various types of energy or energy modalities include, but are not limited to, RF monopolar 366, RF bipolar 368, ultrasonic 370, microwave 372, IRE 374 as shown in FIGS. 6B and 18. Some examples of different types of user defined algorithms 344 are given below.

A user may decide to create a user-defined algorithm 344 by selecting any of the pre-defined algorithms 376, especially for defining an algorithm with sequenced energy modalities (e.g., sequential energy algorithm 358 in FIGS. 6B and 18) that reduces the number of button switch activations and/or mode switching that the user has to perform to complete a desired task. A user-defined algorithm 344 is particularly useful for customization from user to user by varying the properties 378 discussed above. Each user has its own preferences and its own strategy for completing a surgical procedure. For tasks that may be repetitive and may occur several times during a surgery, a user-defined algorithm 344 can be established to reduce the number of actions that the user has to perform during the surgical procedure to complete their target task. Two examples of this are given below.

By way of a first example, a user-defined algorithm 344 may be defined to execute instructions for double cycle RF bipolar sealing and ultrasonic cutting. Accordingly, for extra confidence in a seal on a vessel or for extra debulking of tissue prior to a cut, the user may desire two cycles of RF bipolar energy before a cut sequence (i.e., ultrasonic activation). The user could create a user-defined algorithm 344 that cycles through two RF bipolar activations and an ultrasonic activation with a single button switch press. For the user-defined algorithm 344, the user could have the option to pre-set target energy parameters (e.g., ultrasonic power level or voltage driving the ultrasonic transducer) when the user-defined algorithm 344 is defined, or could elect to change the input parameters at the user Interface during the surgical procedure before activating the user-defined algorithm 344.

By way of a second example, a user-defined algorithm 344 may be defined to execute instructions for RF bipolar sealing and RF monopolar cutting. Accordingly, a user may desire an RF bipolar seal cycle prior to an RF monopolar cut, and may decide to create a user-defined algorithm 344 to reduce the number of button switch activations or mode-switching required to complete this sequence, especially if there are multiple occasions within a surgical procedure that the User would want to complete this given sequence of outputs.

By way of a third example, a user-defined algorithm 344 may be defined to execute instructions for tissue-specific RF bipolar activation and ultrasonic cutting. In a liver-specific surgical procedure, a user may desire to activate a seal-only cycle (i.e., RF bipolar) at low power over an extended period of time under a light clamp on the target tissue in order to debulk the target tissue and achieve more confidence in sealing of very small vessels running through the target tissue. The cut then could be completed with ultrasonic activation. A user-defined algorithm 344 in this scenario would enable the user to pre-set parameters such as target power output and time of activation for the sealing cycle before switching to an ultrasonic cutting cycle. This user-defined algorithm 344 is particularly useful for repetitive activations on sensitive tissues during a surgical procedure.

The surgical instruments 100, 150, 190 and associated algorithms described above in connection with FIGS. 1-21 may be implemented in the following surgical hub system in conjunction with the following generator and modular energy system, for example.

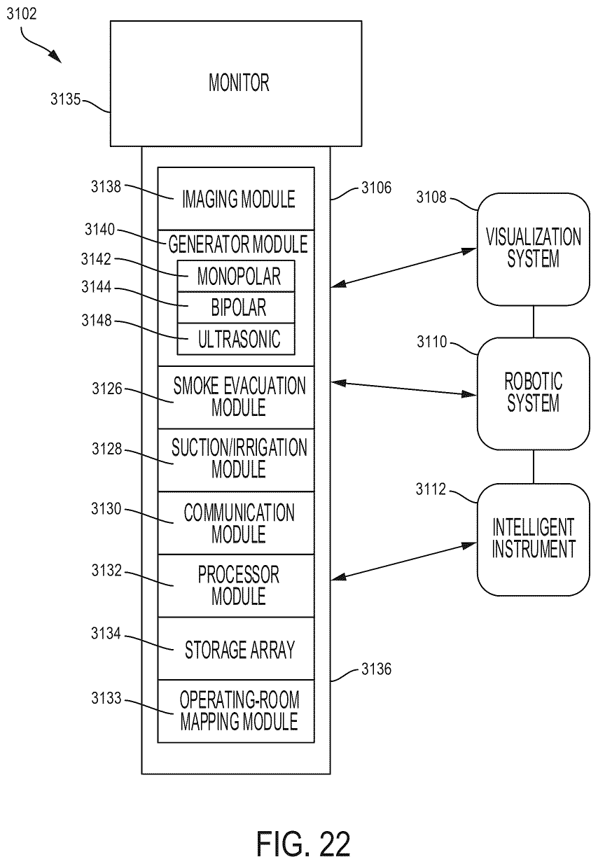

FIG. 22 is a surgical system 3102 comprising a surgical hub 3106 paired with a visualization system 3108, a robotic system 3110, and an intelligent instrument 3112, in accordance with at least one aspect of the present disclosure. Referring now to FIG. 22, the hub 3106 is depicted in communication with a visualization system 3108, a robotic system 3110, and a handheld intelligent surgical instrument 3112 configured in a similar manner to the surgical instruments 100, 150, 190 as described in FIGS. 1-5. The hub 3106 includes a hub display 3135, an imaging module 3138, a generator module 3140, a communication module 3130, a processor module 3132, and a storage array 3134. In certain aspects, as illustrated in FIG. 22, the hub 3106 further includes a smoke evacuation module 3126 and/or a suction/irrigation module 3128.

During a surgical procedure, energy application to tissue, for sealing and/or cutting, is generally associated with smoke evacuation, suction of excess fluid, and/or irrigation of the tissue. Fluid, power, and/or data lines from different sources are often entangled during the surgical procedure. Valuable time can be lost addressing this issue during a surgical procedure. Detangling the lines may necessitate disconnecting the lines from their respective modules, which may require resetting the modules. The hub modular enclosure 3136 offers a unified environment for managing the power, data, and fluid lines, which reduces the frequency of entanglement between such lines.

Aspects of the present disclosure present a surgical hub for use in a surgical procedure that involves energy application to tissue at a surgical site. The surgical hub includes a hub enclosure and a combo generator module slidably receivable in a docking station of the hub enclosure. The docking station includes data and power contacts. The combo generator module includes two or more of an ultrasonic energy generator component, a bipolar RF energy generator component, and a monopolar RF energy generator component that are housed in a single unit. In one aspect, the combo generator module also includes a smoke evacuation component, at least one energy delivery cable for connecting the combo generator module to a surgical instrument, at least one smoke evacuation component configured to evacuate smoke, fluid, and/or particulates generated by the application of therapeutic energy to the tissue, and a fluid line extending from the remote surgical site to the smoke evacuation component.

In one aspect, the fluid line is a first fluid line and a second fluid line extends from the remote surgical site to a suction and irrigation module slidably received in the hub enclosure. In one aspect, the hub enclosure comprises a fluid interface.

Certain surgical procedures may require the application of more than one energy type to the tissue. One energy type may be more beneficial for cutting the tissue, while another different energy type may be more beneficial for sealing the tissue. For example, a bipolar generator can be used to seal the tissue while an ultrasonic generator can be used to cut the sealed tissue. Aspects of the present disclosure present a solution where a hub modular enclosure 136 is configured to accommodate different generators, and facilitate an interactive communication therebetween. One of the advantages of the hub modular enclosure 136 is enabling the quick removal and/or replacement of various modules.

Aspects of the present disclosure present a modular surgical enclosure for use in a surgical procedure that involves energy application to tissue. The modular surgical enclosure includes a first energy-generator module, configured to generate a first energy for application to the tissue, and a first docking station comprising a first docking port that includes first data and power contacts, wherein the first energy-generator module is slidably movable into an electrical engagement with the power and data contacts and wherein the first energy-generator module is slidably movable out of the electrical engagement with the first power and data contacts,

Further to the above, the modular surgical enclosure also includes a second energy-generator module configured to generate a second energy, different than the first energy, for application to the tissue, and a second docking station comprising a second docking port that includes second data and power contacts, wherein the second energy-generator module is slidably movable into an electrical engagement with the power and data contacts, and wherein the second energy-generator module is slidably movable out of the electrical engagement with the second power and data contacts.

In addition, the modular surgical enclosure also includes a communication bus between the first docking port and the second docking port, configured to facilitate communication between the first energy-generator module and the second energy-generator module.

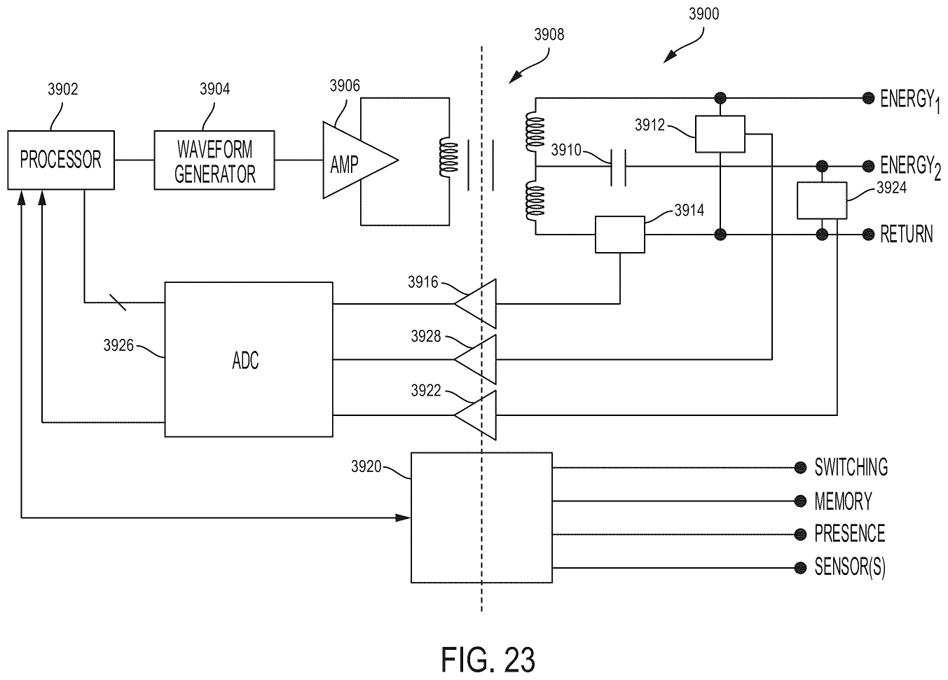

FIG. 23 illustrates an example of a generator 3900, in accordance with at least one aspect of the present disclosure. As shown in FIG. 23, the generator 3900 is one form of a generator configured to couple to a surgical instrument 100, 150, 190 as described in FIGS. 1-5, and further configured to execute adaptive ultrasonic and electrosurgical control algorithms in a surgical data network comprising a modular communication hub as shown in FIG. 22. The generator 3900 is configured to deliver multiple energy modalities to a surgical instrument. The generator 3900 provides RF and ultrasonic signals for delivering energy to a surgical instrument either independently or simultaneously. The RF and ultrasonic signals may be provided alone or in combination and may be provided simultaneously. As noted above, at least one generator output can deliver multiple energy modalities (e.g., ultrasonic, bipolar or monopolar RF, irreversible and/or reversible electroporation, and/or microwave energy, among others) through a single port, and these signals can be delivered separately or simultaneously to the end effector to treat tissue.

The generator 3900 comprises a processor 3902 coupled to a waveform generator 3904. The processor 3902 and waveform generator 3904 are configured to generate a variety of signal waveforms based on information stored in a memory coupled to the processor 3902, not shown for clarity of disclosure. The digital information associated with a waveform is provided to the waveform generator 3904 which includes one or more DAC circuits to convert the digital input into an analog output. The analog output is fed to an amplifier 3906 for signal conditioning and amplification. The conditioned and amplified output of the amplifier 3906 is coupled to a power transformer 3908. The signals are coupled across the power transformer 3908 to the secondary side, which is in the patient isolation side. A first signal of a first energy modality is provided to the surgical instrument between the terminals labeled ENERGY1 and RETURN. A second signal of a second energy modality is coupled across a capacitor 3910 and is provided to the surgical instrument between the terminals labeled ENERGY2 and RETURN. It will be appreciated that more than two energy modalities may be output and thus the subscript “n” may be used to designate that up to n ENERGYn terminals may be provided, where n is a positive integer greater than 1. It also will be appreciated that up to “n” return paths RETURNn may be provided without departing from the scope of the present disclosure.

A first voltage sensing circuit 3912 is coupled across the terminals labeled ENERGY1 and the RETURN path to measure the output voltage therebetween. A second voltage sensing circuit 3924 is coupled across the terminals labeled ENERGY2 and the RETURN path to measure the output voltage therebetween. A current sensing circuit 3914 is disposed in series with the RETURN leg of the secondary side of the power transformer 3908 as shown to measure the output current for either energy modality. If different return paths are provided for each energy modality, then a separate current sensing circuit should be provided in each return leg. The outputs of the first and second voltage sensing circuits 3912, 3924 are provided to respective isolation transformers 3916, 3922 and the output of the current sensing circuit 3914 is provided to another isolation transformer 3918. The outputs of the isolation transformers 3916, 3928, 3922 in the on the primary side of the power transformer 3908 (non-patient isolated side) are provided to a one or more ADC circuit 3926. The digitized output of the ADC circuit 3926 is provided to the processor 3902 for further processing and computation. The output voltages and output current feedback information can be employed to adjust the output voltage and current provided to the surgical instrument and to compute output impedance, among other parameters. Input/output communications between the processor 3902 and patient isolated circuits is provided through an interface circuit 3920. Sensors also may be in electrical communication with the processor 3902 by way of the interface circuit 3920.

In one aspect, the impedance may be determined by the processor 3902 by dividing the output of either the first voltage sensing circuit 3912 coupled across the terminals labeled ENERGY1/RETURN or the second voltage sensing circuit 3924 coupled across the terminals labeled ENERGY2/RETURN by the output of the current sensing circuit 3914 disposed in series with the RETURN leg of the secondary side of the power transformer 3908. The outputs of the first and second voltage sensing circuits 3912, 3924 are provided to separate isolations transformers 3916, 3922 and the output of the current sensing circuit 3914 is provided to another isolation transformer 3916. The digitized voltage and current sensing measurements from the ADC circuit 3926 are provided the processor 3902 for computing impedance. As an example, the first energy modality ENERGY1 may be ultrasonic energy and the second energy modality ENERGY2 may be RF energy. Nevertheless, in addition to ultrasonic and bipolar or monopolar RF energy modalities, other energy modalities include irreversible and/or reversible electroporation and/or microwave energy, among others. Also, although the example illustrated in FIG. 23 shows a single return path RETURN may be provided for two or more energy modalities, in other aspects, multiple return paths RETURNn may be provided for each energy modality ENERGYn. Thus, as described herein, the ultrasonic transducer impedance may be measured by dividing the output of the first voltage sensing circuit 3912 by the current sensing circuit 3914 and the tissue impedance may be measured by dividing the output of the second voltage sensing circuit 3924 by the current sensing circuit 3914.

As shown in FIG. 23, the generator 3900 comprising at least one output port can include a power transformer 3908 with a single output and with multiple taps to provide power in the form of one or more energy modalities, such as ultrasonic, bipolar or monopolar RF, irreversible and/or reversible electroporation, and/or microwave energy, among others, for example, to the end effector depending on the type of treatment of tissue being performed. For example, the generator 3900 can deliver energy with higher voltage and lower current to drive an ultrasonic transducer, with lower voltage and higher current to drive RF electrodes for sealing tissue, or with a coagulation waveform for spot coagulation using either monopolar or bipolar RF electrosurgical electrodes. The output waveform from the generator 3900 can be steered, switched, or filtered to provide the frequency to the end effector of the surgical instrument. The connection of an ultrasonic transducer to the generator 3900 output would be preferably located between the output labeled ENERGY1 and RETURN as shown in FIG. 23. In one example, a connection of RF bipolar electrodes to the generator 3900 output would be preferably located between the output labeled ENERGY2 and RETURN. In the case of monopolar output, the preferred connections would be active electrode (e.g., pencil or other probe) to the ENERGY2 output and a suitable return pad connected to the RETURN output.

Additional details are disclosed in U.S. Patent Application Publication No. 2017/0086914, titled TECHNIQUES FOR OPERATING GENERATOR FOR DIGITALLY GENERATING ELECTRICAL SIGNAL WAVEFORMS AND SURGICAL INSTRUMENTS, which published on Mar. 30, 2017, which is herein incorporated by reference in its entirety.

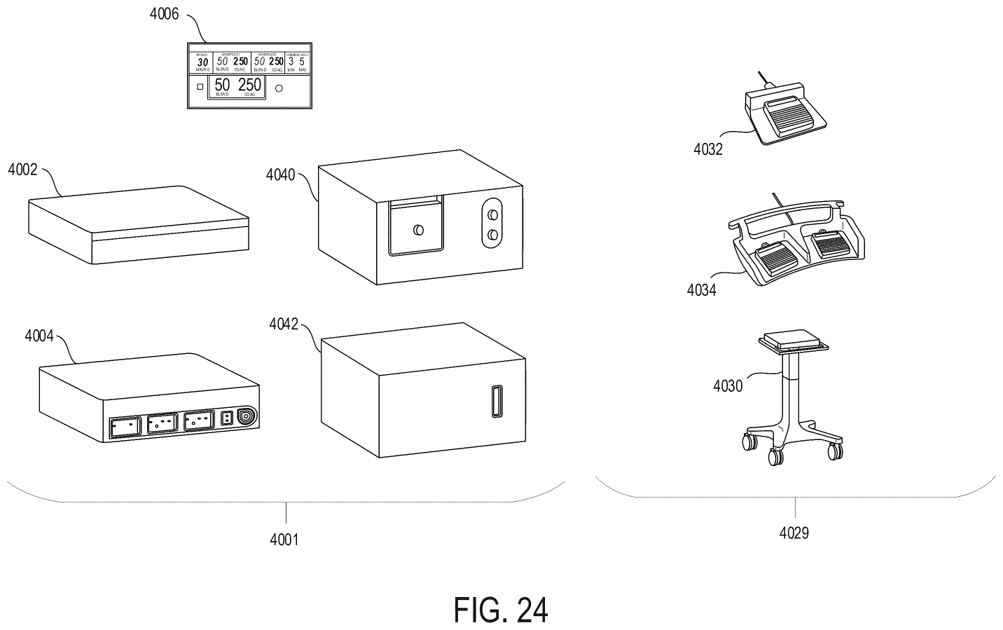

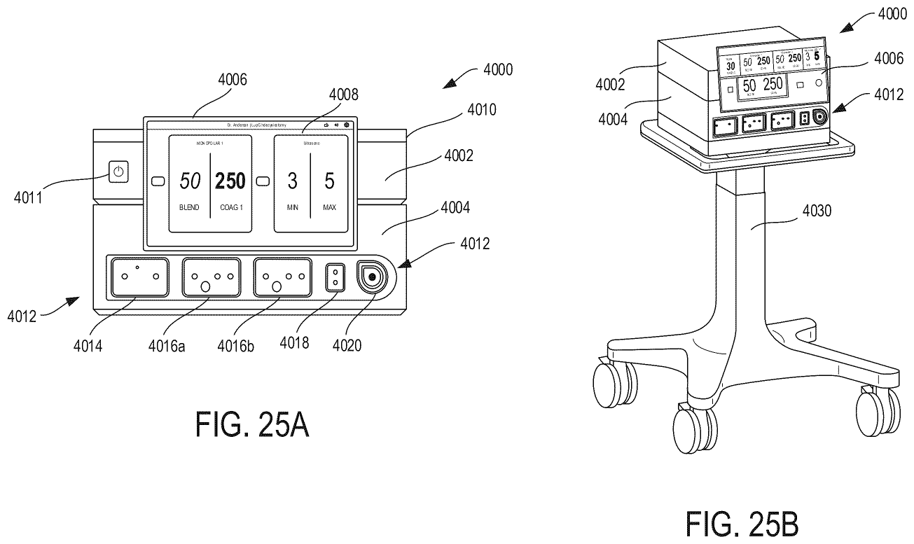

FIG. 24 is a diagram of various modules and other components that are combinable to customize modular energy systems, in accordance with at least one aspect of the present disclosure. FIG. 25A is a first illustrative modular energy system configuration including a header module and a display screen that renders a graphical user interface (GUI) for relaying information regarding modules connected to the header module, in accordance with at least one aspect of the present disclosure. FIG. 25B is the modular energy system shown in FIG. 25A mounted to a cart, in accordance with at least one aspect of the present disclosure.

With reference now to FIGS. 24-25B, ORs everywhere in the world are a tangled web of cords, devices, and people due to the amount of equipment required to perform surgical procedures. Surgical capital equipment tends to be a major contributor to this issue because most surgical capital equipment performs a single, specialized task. Due to their specialized nature and the surgeons' needs to utilize multiple different types of devices during the course of a single surgical procedure, an OR may be forced to be stocked with two or even more pieces of surgical capital equipment, such as energy generators. Each of these pieces of surgical capital equipment must be individually plugged into a power source and may be connected to one or more other devices that are being passed between OR personnel, creating a tangle of cords that must be navigated. Another issue faced in modern ORs is that each of these specialized pieces of surgical capital equipment has its own user interface and must be independently controlled from the other pieces of equipment within the OR. This creates complexity in properly controlling multiple different devices in connection with each other and forces users to be trained on and memorize different types of user interfaces (which may further change based upon the task or surgical procedure being performed, in addition to changing between each piece of capital equipment). This cumbersome, complex process can necessitate the need for even more individuals to be present within the OR and can create danger if multiple devices are not properly controlled in tandem with each other. Therefore, consolidating surgical capital equipment technology into singular systems that are able to flexibly address surgeons' needs to reduce the footprint of surgical capital equipment within ORs would simplify the user experience, reduce the amount of clutter in ORs, and prevent difficulties and dangers associated with simultaneously controlling multiple pieces of capital equipment. Further, making such systems expandable or customizable would allow for new technology to be conveniently incorporated into existing surgical systems, obviating the need to replace entire surgical systems or for OR personnel to learn new user interfaces or equipment controls with each new technology.

A surgical hub can be configured to interchangeably receive a variety of modules, which can in turn interface with surgical devices (e.g., a surgical instrument or a smoke evacuator) or provide various other functions (e.g., communications). In one aspect, a surgical hub can be embodied as a modular energy system 4000, which is illustrated in connection with FIGS. 24-25B. The modular energy system 4000 can include a variety of different modules 4001 that are connectable together in a stacked configuration. In one aspect, the modules 4001 can be both physically and communicably coupled together when stacked or otherwise connected together into a singular assembly. Further, the modules 4001 can be interchangeably connectable together in different combinations or arrangements. In one aspect, each of the modules 4001 can include a consistent or universal array of connectors disposed along their upper and lower surfaces, thereby allowing any module 4001 to be connected to another module 4001 in any arrangement (except that, in some aspects, a particular module type, such as the header module 4002, can be configured to serve as the uppermost module within the stack, for example). In an alternative aspect, the modular energy system 4000 can include a housing that is configured to receive and retain the modules 4001, as is shown in FIG. 22. The modular energy system 4000 can also include a variety of different components or accessories that are also connectable to or otherwise associatable with the modules 4001. In another aspect, the modular energy system 4000 can be embodied as a generator module 3140, 3900 (FIGS. 22-23) of a surgical hub 3106. In yet another aspect, the modular energy system 4000 can be a distinct system from a surgical hub 3106. In such aspects, the modular energy system 4000 can be communicably couplable to a surgical hub 3106 for transmitting and/or receiving data therebetween.

The modular energy system 4000 can be assembled from a variety of different modules 4001, some examples of which are illustrated in FIG. 24. Each of the different types of modules 4001 can provide different functionality, thereby allowing the modular energy system 4000 to be assembled into different configurations to customize the functions and capabilities of the modular energy system 4000 by customizing the modules 4001 that are included in each modular energy system 4000. The modules 4001 of the modular energy system 4000 can include, for example, a header module 4002 (which can include a display screen 4006), an energy module 4004, a technology module 4040, and a visualization module 4042. In the depicted aspect, the header module 4002 is configured to serve as the top or uppermost module within the modular energy system stack and can thus lack connectors along its top surface. In another aspect, the header module 4002 can be configured to be positioned at the bottom or the lowermost module within the modular energy system stack and can thus lack connectors along its bottom surface. In yet another aspect, the header module 4002 can be configured to be positioned at an intermediate position within the modular energy system stack and can thus include connectors along both its bottom and top surfaces.

The header module 4002 can be configured to control the system-wide settings of each module 4001 and component connected thereto through physical controls 4011 thereon and/or a graphical user interface (GUI) 4008 rendered on the display screen 4006. Such settings could include the activation of the modular energy system 4000, the volume of alerts, the footswitch settings, the settings icons, the appearance or configuration of the user interface, the surgeon profile logged into the modular energy system 4000, and/or the type of surgical procedure being performed. The header module 4002 can also be configured to provide communications, processing, and/or power for the modules 4001 that are connected to the header module 4002. The energy module 4004, which can also be referred to as a generator module 3140, 3900 (FIGS. 22-23), can be configured to generate one or multiple energy modalities for driving electrosurgical and/or ultrasonic surgical instruments connected thereto, such as is described above in connection with the generator 3900 illustrated in FIG. 23. The technology module 4040 can be configured to provide additional or expanded control algorithms (e.g., electrosurgical or ultrasonic control algorithms for controlling the energy output of the energy module 4004). The visualization module 4042 can be configured to interface with visualization devices (i.e., scopes) and accordingly provide increased visualization capabilities.

The modular energy system 4000 can further include a variety of accessories 4029 that are connectable to the modules 4001 for controlling the functions thereof or that are otherwise configured to work on conjunction with the modular energy system 4000. The accessories 4029 can include, for example, a single-pedal footswitch 4032, a dual-pedal footswitch 4034, and a cart 4030 for supporting the modular energy system 4000 thereon. The footswitches 4032, 4034 can be configured to control the activation or function of particular energy modalities output by the energy module 4004, for example.

By utilizing modular components, the depicted modular energy system 4000 provides a surgical platform that grows with the availability of technology and is customizable to the needs of the facility and/or surgeons. Further, the modular energy system 4000 supports combo devices (e.g., dual electrosurgical and ultrasonic energy generators) and supports software-driven algorithms for customized tissue effects. Still further, the surgical system architecture reduces the capital footprint by combining multiple technologies critical for surgery into a single system.

The various modular components utilizable in connection with the modular energy system 4000 can include monopolar energy generators, bipolar energy generators, dual electrosurgical/ultrasonic energy generators, display screens, and various other modules and/or other components, some of which are also described above in connection with FIGS. 1-21.

Referring now to FIG. 25A, the header module 4002 can, in some aspects, include a display screen 4006 that renders a GUI 4008 for relaying information regarding the modules 4001 connected to the header module 4002. In some aspects, the GUI 4008 of the display screen 4006 can provide a consolidated point of control of all of the modules 4001 making up the particular configuration of the modular energy system 4000. In alternative aspects, the header module 4002 can lack the display screen 4006 or the display screen 4006 can be detachably connected to the housing 4010 of the header module 4002. In such aspects, the header module 4002 can be communicably couplable to an external system that is configured to display the information generated by the modules 4001 of the modular energy system 4000. For example, in robotic surgical applications, the modular energy system 4000 can be communicably couplable to a robotic cart or robotic control console, which is configured to display the information generated by the modular energy system 4000 to the operator of the robotic surgical system. As another example, the modular energy system 4000 can be communicably couplable to a mobile display that can be carried or secured to a surgical staff member for viewing thereby. In yet another example, the modular energy system 4000 can be communicably couplable to a surgical hub 4100 or another computer system that can include a display 4104. In aspects utilizing a user interface that is separate from or otherwise distinct from the modular energy system 4000, the user interface can be wirelessly connectable with the modular energy system 4000 as a whole or one or more modules 4001 thereof such that the user interface can display information from the connected modules 4001 thereon.

Referring still to FIG. 25A, the energy module 4004 can include a port assembly 4012 including a number of different ports configured to deliver different energy modalities to corresponding surgical instruments that are connectable thereto. In the particular aspect illustrated in FIGS. 24-25B, the port assembly 4012 includes a bipolar port 4014, a first monopolar port 4016a, a second monopolar port 4018b, a neutral electrode port 4018 (to which a monopolar return pad is connectable), and a combination energy port 4020. However, this particular combination of ports is simply provided for illustrative purposes and alternative combinations of ports and/or energy modalities may be possible for the port assembly 4012.

As noted above, the modular energy system 4000 can be assembled into different configurations. Further, the different configurations of the modular energy system 4000 can also be utilizable for different surgical procedure types and/or different tasks. For example, FIGS. 25A and 25B illustrate a first illustrative configuration of the modular energy system 4000 including a header module 4002 (including a display screen 4006) and an energy module 4004 connected together. Such a configuration can be suitable for laparoscopic and open surgical procedures, for example.

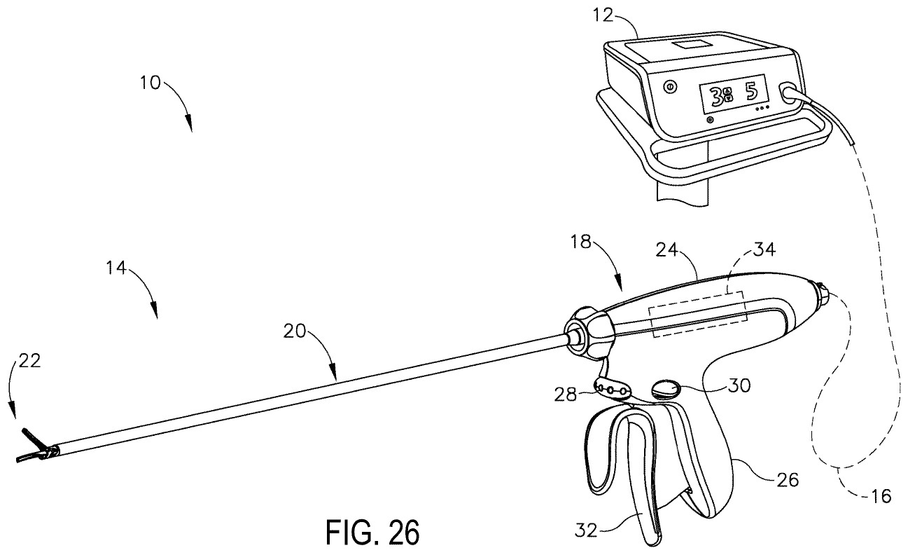

FIGS. 26-30 illustrate an example surgical system 10 with ultrasonic and electrosurgical features including any one of the end-effectors, surgical instruments, and generators described herein. FIG. 26 depicts a surgical system 10 including a generator 12 and a surgical instrument 14. The surgical instrument 14 is operatively coupled with the generator 12 via a power cable 16. The generator 12 is operable to power the surgical instrument 14 to deliver ultrasonic energy for cutting tissue, and electrosurgical bipolar RF energy (i.e., therapeutic levels of RF energy) for sealing tissue. In one aspect, the generator 12 is configured to power the surgical instrument 14 to deliver ultrasonic energy and electrosurgical bipolar RF energy simultaneously or independently.

The surgical instrument 14 of the present example comprises a handle assembly 18, a shaft assembly 20 extending distally from the handle assembly 18, and an end effector 22 arranged at a distal end of the shaft assembly 20. The handle assembly 18 comprises a body 24 including a pistol grip 26 and energy control buttons 28, 30 configured to be manipulated by a surgeon. A trigger 32 is coupled to a lower portion of the body 24 and is pivotable toward and away from the pistol grip 26 to selectively actuate the end effector 22, as described in greater detail below. In other suitable variations of the surgical instrument 14, the handle assembly 18 may comprise a scissor grip configuration, for example. An ultrasonic transducer 34 is housed internally within and supported by the body 24. In other configurations, the ultrasonic transducer 34 may be provided externally of the body 24.

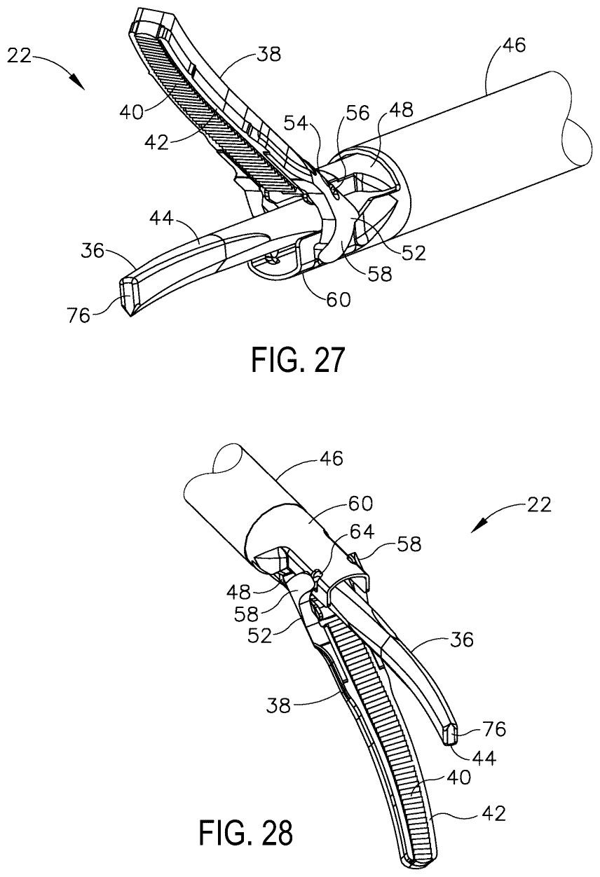

As shown in FIGS. 27 and 28, the end effector 22 includes an ultrasonic blade 36 and a clamp arm 38 configured to selectively pivot toward and away from the ultrasonic blade 36, for clamping tissue therebetween. The ultrasonic blade 36 is acoustically coupled with the ultrasonic transducer 34, which is configured to drive (i.e., vibrate) the ultrasonic blade 36 at ultrasonic frequencies for cutting and/or sealing tissue positioned in contact with the ultrasonic blade 36. The clamp arm 38 is operatively coupled with the trigger 32 such that the clamp arm 38 is configured to pivot toward the ultrasonic blade 36, to a closed position, in response to pivoting of the trigger 32 toward the pistol grip 26. Further, the clamp arm 38 is configured to pivot away from the ultrasonic blade 36, to an open position (see e.g., FIGS. 26-28), in response to pivoting of the trigger 32 away from the pistol grip 26. Various suitable ways in which the clamp arm 38 may be coupled with the trigger 32 will be apparent to those of ordinary skill in the art in view of the teachings provided herein. In some versions, one or more resilient members may be incorporated to bias the clamp arm 38 and/or the trigger 32 toward the open position.

A clamp pad 40 is secured to and extends distally along a clamping side of the clamp arm 38, facing the ultrasonic blade 36. The clamp pad 40 is configured to engage and clamp tissue against a corresponding tissue treatment portion of the ultrasonic blade 36 when the clamp arm 38 is actuated to its closed position. At least a clamping-side of the clamp arm 38 provides a first electrode 42, referred to herein as clamp arm electrode 42. Additionally, at least a clamping-side of the ultrasonic blade 36 provides a second electrode 44, referred to herein as a blade electrode 44. The electrodes 42, 44 are configured to apply electrosurgical bipolar RF energy, provided by the generator 12, to tissue electrically coupled with the electrodes 42, 44. The clamp arm electrode 42 may serve as an active electrode while the blade electrode 44 serves as a return electrode, or vice-versa. The surgical instrument 14 may be configured to apply the electrosurgical bipolar RF energy through the electrodes 42, 44 while vibrating the ultrasonic blade 36 at an ultrasonic frequency, before vibrating the ultrasonic blade 36 at an ultrasonic frequency, and/or after vibrating the ultrasonic blade 36 at an ultrasonic frequency.

As shown in FIGS. 26-30, the shaft assembly 20 extends along a longitudinal axis and includes an outer tube 46, an inner tube 48 received within the outer tube 46, and an ultrasonic waveguide 50 supported within the inner tube 48. As seen best in FIGS. 27-30, the clamp arm 38 is coupled to distal ends of the inner and outer tubes 46, 48. In particular, the clamp arm 38 includes a pair of proximally extending clevis arms 52 that receive therebetween and pivotably couple to a distal end 54 of the inner tube 48 with a pivot pin 56 received through bores formed in the clevis arms 52 and the distal end 54 of the inner tube 48. The first and second clevis fingers 58 depend downwardly from the clevis arms 52 and pivotably couple to a distal end 60 of the outer tube 46. Specifically, each clevis finger 58 includes a protrusion 62 that is rotatably received within a corresponding opening 64 formed in a sidewall of the distal end 60 of the outer tube 46.

In the present example, the inner tube 48 is longitudinally fixed relative to the handle assembly 18, and the outer tube 46 is configured to translate relative to the inner tube 48 and the handle assembly 18, along the longitudinal axis of the shaft assembly 20. As the outer tube 46 translates distally, the clamp arm 38 pivots about the pivot pin 56 toward its open position. As the outer tube 46 translates proximally, the clamp arm 38 pivots in an opposite direction toward its closed position. A proximal end of the outer tube 46 is operatively coupled with the trigger 32, for example via a linkage assembly, such that actuation of the trigger 32 causes translation of the outer tube 46 relative to the inner tube 48, thereby opening or closing the clamp arm 38. In other suitable configurations not shown herein, the outer tube 46 may be longitudinally fixed and the inner tube 48 may be configured to translate for moving the clamp arm 38 between its open and closed positions.

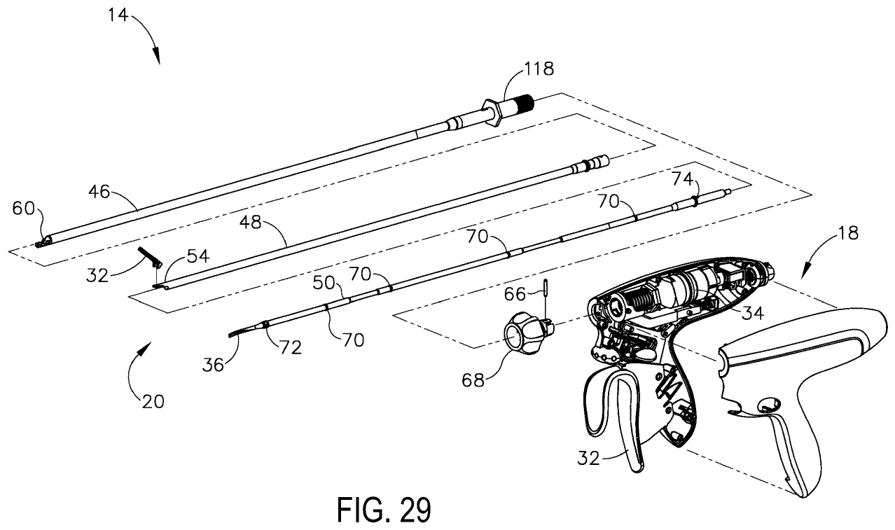

The shaft assembly 20 and the end effector 22 are configured to rotate together about the longitudinal axis, relative to the handle assembly 18. A retaining pin 66, shown in FIG. 29, extends transversely through the proximal portions of the outer tube 46, the inner tube 48, and the waveguide 50 to thereby couple these components rotationally relative to one another. In the present example, a rotation knob 68 is provided at a proximal end portion of the shaft assembly 20 to facilitate rotation of the shaft assembly 20, and the end effector 22, relative to the handle assembly 18. The rotation knob 68 is secured rotationally to the shaft assembly 20 with the retaining pin 66, which extends through a proximal collar of the rotation knob 68. It will be appreciated that in other suitable configurations, the rotation knob 6) may be omitted or substituted with alternative rotational actuation structures.

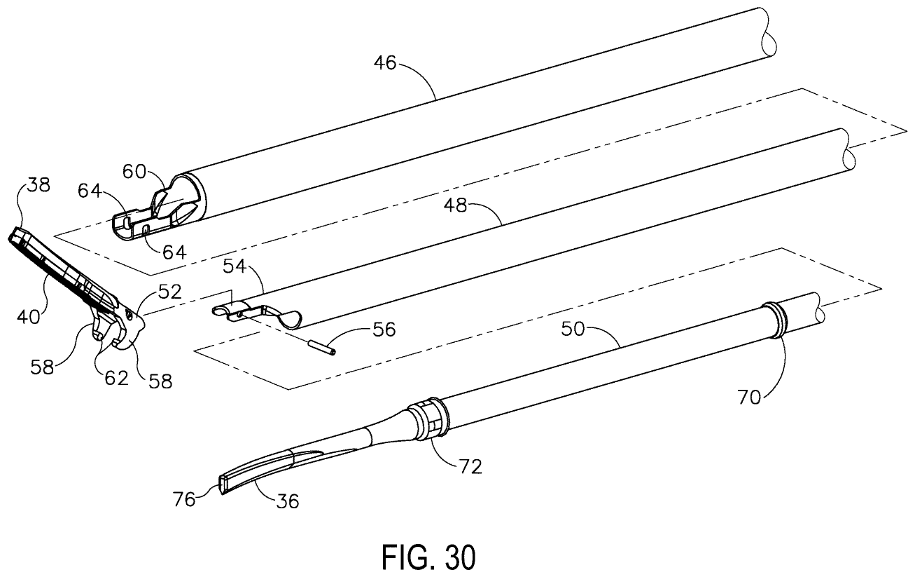

The ultrasonic waveguide 50 is acoustically coupled at its proximal end with the ultrasonic transducer 34, for example by a threaded connection, and at its distal end with the ultrasonic blade 36, as shown in FIG. 30. The ultrasonic blade 36 is shown formed integrally with the waveguide 50 such that the blade 36 extends distally, directly from the distal end of the waveguide 50. In this manner, the waveguide 50 acoustically couples the ultrasonic transducer 34 with the ultrasonic blade 36, and functions to communicate ultrasonic mechanical vibrations from the transducer 34 to the blade 36. Accordingly, the ultrasonic transducer 34, the waveguide 50, and the ultrasonic blade 36 together define an acoustic assembly. During use, the ultrasonic blade 36 may be positioned in direct contact with tissue, with or without assistive clamping force provided by the clamp arm 38, to impart ultrasonic vibrational energy to the tissue and thereby cut and/or seal the tissue. For example, the blade 36 may cut through tissue clamped between the clamp arm 38 and a first treatment side of the blade 36, or the blade 36 may cut through tissue positioned in contact with an oppositely disposed second treatment side of the blade 36, for example during a “back-cutting” movement. In some variations, the waveguide 50 may amplify the ultrasonic vibrations delivered to the blade 36. Further, the waveguide 50 may include various features operable to control the gain of the vibrations, and/or features suitable to tune the waveguide 50 to a selected resonant frequency. Additional features of the ultrasonic blade 36 and the waveguide 50 are described in greater detail below.

The waveguide 50 is supported within the inner tube 48 by a plurality of nodal support elements 70 positioned along a length of the waveguide 50, as shown in FIGS. 29-30. Specifically, the nodal support elements 70 are positioned longitudinally along the waveguide 50 at locations corresponding to acoustic nodes defined by the resonant ultrasonic vibrations communicated through the waveguide 50. The nodal support elements 70 may provide structural support to the waveguide 50, and acoustic isolation between the waveguide 50 and the inner and outer tubes 46, 48 of the shaft assembly 20. In variations, the nodal support elements 70 may comprise o-rings. The waveguide 50 is supported at its distal-most acoustic node by a nodal support element in the form of an overmold member 72, shown in FIG. 30. The waveguide 50 is secured longitudinally and rotationally within the shaft assembly 20 by the retaining pin 66, which passes through a transverse through-bore 74 formed at a proximally arranged acoustic node of the waveguide 50, such as the proximal-most acoustic node, for example.

In the present example, a distal tip 76 of the ultrasonic blade 36 is located at a position corresponding to an anti-node associated with the resonant ultrasonic vibrations communicated through the waveguide 50. Such a configuration enables the acoustic assembly of the instrument 14 to be tuned to a preferred resonant frequency fₒ when the ultrasonic blade 36 is not loaded by tissue. When the ultrasonic transducer 34 is energized by the generator 12 to transmit mechanical vibrations through the waveguide 50 to the blade 36, the distal tip 76 of the blade 36 is caused to oscillate longitudinally in the range of approximately 20 to 120 microns peak-to-peak, for example, and in some instances in the range of approximately 20 to 50 microns, at a predetermined vibratory frequency fₒ of approximately 50 kHz, for example. When the ultrasonic blade 36 is positioned in contact with tissue, the ultrasonic oscillation of the blade 36 may simultaneously sever the tissue and denature the proteins in adjacent tissue cells, thereby providing a coagulative effect with minimal thermal spread.

### EXAMPLES

Examples of the method according to various aspects of the present disclosure are provided below. An aspect of the method may include any one or more than one, and any combination of, the examples described below.

Example 1. The present disclosure provides a surgical instrument. The surgical instrument comprises a housing, an end-effector, and a user interface. The end-effector comprises a clamp arm and an ultrasonic blade configured to couple to an ultrasonic transducer and to a pole of an electrical generator. The clamp arm comprises a clamp jaw pivotally movable about a pivot point and an electrode defining a surface configured to contact tissue and apply electrical energy to the tissue in contact therewith. The electrode is configured to couple to an opposite pole of the electrical generator. The user interface comprises a first activation button switch to activate a first energy source and a second button switch to select an energy mode for the activation button switch.

Example 2. The surgical instrument of Example 1, wherein the user interface further comprises a closure trigger to operate the end-effector; and a switch to activate a second energy source.

Example 3. The surgical instrument of any one of Examples 1-2, wherein the second button switch is a separate control from the end effector closure trigger.

Example 4. The surgical instrument of any one of Examples 1-3, wherein the closure trigger comprises a two stage activation, wherein the second button switch is configured to activate in a second stage of the closure trigger.

Example 5. The surgical instrument of any one of Examples 1-4, wherein a first stage of the closure trigger is configured to activate in the closing direction to actuate the end-effector.

Example 6. The surgical instrument of any one of Examples 1-5, wherein the user interface further comprises a rotatable knob to rotate a shaft and the end-effector coupled to the shaft.

Example 7. The surgical instrument of any one of Examples 1-6, wherein the second button switch is coupled to a circuit comprising at least one input parameter to define the energy mode.

Example 8. The surgical instrument of any one of Examples 1-7, wherein the input parameter is remotely configured through connection to a generator or through a software update.

Example 9. The surgical instrument of any one of Examples 1-8, wherein the input parameter is either duty cycle, voltage, frequency, pulse width, or current, or any combination thereof.

Example 10. The surgical instrument of any one of Examples 1-9, wherein the input parameter is at least one of energy type, duty cycle, voltage, frequency, pulse width, current, impedance limit, activation time, or blend of energy, or any combination thereof.

Example 11. The surgical instrument of any one of Examples 1-10, wherein the second button switch is configured to select from a list of predefined modes and the number of modes in the list is either predefined or defined by a second input parameter defined by a user.

Example 12. The surgical instrument of any one of Examples 1-11, wherein at least one of the energy modes is a simultaneous blend of RF and ultrasonic energy, and the input parameter represents a duty cycle of the RF and ultrasonic energy.

Example 13. The surgical instrument of any one of Examples 1-12, wherein the second button switch is configured to select from a list of predefined modes and the number of modes in the list is defined by a second input parameter defined by a user.

Example 14. The surgical instrument of any one of Examples 1-13, wherein the input parameter is either duty cycle, voltage, frequency, pulse width, or current, or any combination thereof.

Example 15. The surgical instrument of any one of Examples 1-14, wherein the user interface further comprises a visual indicator of the selected energy mode disposed on the housing.

Example 16. The surgical instrument of any one of Examples 1-15, wherein the user interface further comprises a visual indicator of the selected energy mode disposed on a portion of a shaft coupling the housing and the end-effector, wherein the visual indicator is located in a surgical field of view.

Example 17. The surgical instrument of any one of Examples 1-16, wherein at least one of the energy modes is selected from ultrasonic, RF bipolar, RF monopolar, microwave, or IRE, or any combination thereof.

Example 18. The surgical instrument of any one of Examples 1-17, wherein at least one of the energy modes is configured apply energy based on a predefined duty cycle or pulsed algorithm.

Example 19. The surgical instrument of any one of Examples 1-18, wherein at least one of the energy modes is selected from a sequential application of two or more of ultrasonic, RF bipolar, RF monopolar, microwave, or IRE energy types, or any combination thereof.

Example 20. The surgical instrument of any one of Examples 1-19, wherein at least one of the energy modes is a simultaneous blend of two or more of ultrasonic, RF bipolar, RF monopolar, microwave, or IRE energy types.

Example 21. The surgical instrument of any one of Examples 1-20, wherein at least one of the energy modes is a simultaneous blend of two or more of ultrasonic, RF bipolar, RF monopolar, microwave, or IRE energy types followed by sequential application of one or more of the ultrasonic, RF bipolar, RF monopolar, microwave, or IRE energy types, or any combination thereof.

Example 22. The surgical instrument of any one of Examples 1-21, wherein at least one of the energy modes is ultrasonic, RF bipolar, RF monopolar, microwave, or IRE energy types followed by a sequential application of a simultaneous blend of two or more of the ultrasonic, RF bipolar, RF monopolar, microwave, or IRE energy types, or any combination thereof.

Example 23. The surgical instrument of any one of Examples 1-22, wherein at least one of the energy modes delivers energy based on a tissue specific predefined algorithm.

Example 24. The surgical instrument of any one of Examples 1-23, wherein at least one of the energy modes is compiled from learned surgical behaviors or activities.

Example 25. The surgical instrument of any one of Examples 1-24, wherein the energy mode comprises ultrasonic, RF bipolar, RF monopolar, microwave, or IRE energy types and are made available to the user through software updates to a generator coupled to the surgical instrument, or any combination thereof.

Example 26. The surgical instrument of any one of Examples 1-25, wherein the energy mode comprises ultrasonic, RF bipolar, RF monopolar, microwave, or IRE energy types and are made available to the user through software updates to the surgical instrument, or any combination thereof.

Example 27. The surgical instrument of any one of Examples 1-26, wherein preferred selections by the user are made available to multiple generators through either networking, the cloud, or manual transfer, or any combination thereof.

While several forms have been illustrated and described, it is not the intention of Applicant to restrict or limit the scope of the appended claims to such detail. Numerous modifications, variations, changes, substitutions, combinations, and equivalents to those forms may be implemented and will occur to those skilled in the art without departing from the scope of the present disclosure. Moreover, the structure of each element associated with the described forms can be alternatively described as a means for providing the function performed by the element. Also, where materials are disclosed for certain components, other materials may be used. It is therefore to be understood that the foregoing description and the appended claims are intended to cover all such modifications, combinations, and variations as falling within the scope of the disclosed forms. The appended claims are intended to cover all such modifications, variations, changes, substitutions, modifications, and equivalents.

The foregoing detailed description has set forth various forms of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, and/or examples can be implemented, individually and/or collectively, by a wide range of hardware, software, firmware, or virtually any combination thereof. Those skilled in the art will recognize that some aspects of the forms disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as one or more program products in a variety of forms, and that an illustrative form of the subject matter described herein applies regardless of the particular type of signal bearing medium used to actually carry out the distribution.

Instructions used to program logic to perform various disclosed aspects can be stored within a memory in the system, such as dynamic random access memory (DRAM), cache, flash memory, or other storage. Furthermore, the instructions can be distributed via a network or by way of other computer readable media. Thus a machine-readable medium may include any mechanism for storing or transmitting information in a form readable by a machine (e.g., a computer), but is not limited to, floppy diskettes, optical disks, compact disc, read-only memory (CD-ROMs), and magneto-optical disks, read-only memory (ROMs), random access memory (RAM), erasable programmable read-only memory (EPROM), electrically erasable programmable read-only memory (EEPROM), magnetic or optical cards, flash memory, or a tangible, machine-readable storage used in the transmission of information over the Internet via electrical, optical, acoustical or other forms of propagated signals (e.g., carrier waves, infrared signals, digital signals, etc.). Accordingly, the non-transitory computer-readable medium includes any type of tangible machine-readable medium suitable for storing or transmitting electronic instructions or information in a form readable by a machine (e.g., a computer).

As used in any aspect herein, the term “control circuit” may refer to, for example, hardwired circuitry, programmable circuitry (e.g., a computer processor including one or more individual instruction processing cores, processing unit, processor, microcontroller, microcontroller unit, controller, digital signal processor (DSP), programmable logic device (PLD), programmable logic array (PLA), or field programmable gate array (FPGA)), state machine circuitry, firmware that stores instructions executed by programmable circuitry, and any combination thereof. The control circuit may, collectively or individually, be embodied as circuitry that forms part of a larger system, for example, an integrated circuit (IC), an application-specific integrated circuit (ASIC), a system on-chip (SoC), desktop computers, laptop computers, tablet computers, servers, smart phones, etc. Accordingly, as used herein “control circuit” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), and/or electrical circuitry forming a communications device (e.g., a modem, communications switch, or optical-electrical equipment). Those having skill in the art will recognize that the subject matter described herein may be implemented in an analog or digital fashion or some combination thereof.

As used in any aspect herein, the term “logic” may refer to an app, software, firmware and/or circuitry configured to perform any of the aforementioned operations. Software may be embodied as a software package, code, instructions, instruction sets and/or data recorded on non-transitory computer readable storage medium. Firmware may be embodied as code, instructions or instruction sets and/or data that are hard-coded (e.g., nonvolatile) in memory devices.

As used in any aspect herein, the terms “component,” “system,” “module” and the like can refer to a computer-related entity, either hardware, a combination of hardware and software, software, or software in execution.

As used in any aspect herein, an “algorithm” refers to a self-consistent sequence of steps leading to a desired result, where a “step” refers to a manipulation of physical quantities and/or logic states which may, though need not necessarily, take the form of electrical or magnetic signals capable of being stored, transferred, combined, compared, and otherwise manipulated. It is common usage to refer to these signals as bits, values, elements, symbols, characters, terms, numbers, or the like. These and similar terms may be associated with the appropriate physical quantities and are merely convenient labels applied to these quantities and/or states.

A network may include a packet switched network. The communication devices may be capable of communicating with each other using a selected packet switched network communications protocol. One example communications protocol may include an Ethernet communications protocol which may be capable permitting communication using a Transmission Control Protocol/Internet Protocol (TCP/IP). The Ethernet protocol may comply or be compatible with the Ethernet standard published by the Institute of Electrical and Electronics Engineers (IEEE) titled “IEEE 802.3 Standard”, published in December, 2008 and/or later versions of this standard. Alternatively or additionally, the communication devices may be capable of communicating with each other using an X.25 communications protocol. The X.25 communications protocol may comply or be compatible with a standard promulgated by the International Telecommunication Union-Telecommunication Standardization Sector (ITU-T). Alternatively or additionally, the communication devices may be capable of communicating with each other using a frame relay communications protocol. The frame relay communications protocol may comply or be compatible with a standard promulgated by Consultative Committee for International Telegraph and Telephone (CCITT) and/or the American National Standards Institute (ANSI). Alternatively or additionally, the transceivers may be capable of communicating with each other using an Asynchronous Transfer Mode (ATM) communications protocol. The ATM communications protocol may comply or be compatible with an ATM standard published by the ATM Forum titled “ATM-MPLS Network Interworking 2.0” published August 2001, and/or later versions of this standard. Of course, different and/or after-developed connection-oriented network communication protocols are equally contemplated herein.

Unless specifically stated otherwise as apparent from the foregoing disclosure, it is appreciated that, throughout the foregoing disclosure, discussions using terms such as “processing,” “computing,” “calculating,” “determining,” “displaying,” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

One or more components may be referred to herein as “configured to,” “configurable to,” “operable/operative to,” “adapted/adaptable,” “able to,” “conformable/conformed to,” etc. Those skilled in the art will recognize that “configured to” can generally encompass active-state components and/or inactive-state components and/or standby-state components, unless context requires otherwise.

The terms “proximal” and “distal” are used herein with reference to a clinician manipulating the handle portion of the surgical instrument. The term “proximal” refers to the portion closest to the clinician and the term “distal” refers to the portion located away from the clinician. It will be further appreciated that, for convenience and clarity, spatial terms such as “vertical”, “horizontal”, “up”, and “down” may be used herein with respect to the drawings. However, surgical instruments are used in many orientations and positions, and these terms are not intended to be limiting and/or absolute.

Those skilled in the art will recognize that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, those skilled in the art will appreciate that recited operations therein may generally be performed in any order. Also, although various operational flow diagrams are presented in a sequence(s), it should be understood that the various operations may be performed in other orders than those which are illustrated, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

It is worthy to note that any reference to “one aspect,” “an aspect,” “an exemplification,” “one exemplification,” and the like means that a particular feature, structure, or characteristic described in connection with the aspect is included in at least one aspect. Thus, appearances of the phrases “in one aspect,” “in an aspect,” “in an exemplification,” and “in one exemplification” in various places throughout the specification are not necessarily all referring to the same aspect. Furthermore, the particular features, structures or characteristics may be combined in any suitable manner in one or more aspects.

Any patent application, patent, non-patent publication, or other disclosure material referred to in this specification and/or listed in any Application Data Sheet is incorporated by reference herein, to the extent that the incorporated materials is not inconsistent herewith. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

In summary, numerous benefits have been described which result from employing the concepts described herein. The foregoing description of the one or more forms has been presented for purposes of illustration and description. It is not intended to be exhaustive or limiting to the precise form disclosed. Modifications or variations are possible in light of the above teachings. The one or more forms were chosen and described in order to illustrate principles and practical application to thereby enable one of ordinary skill in the art to utilize the various forms and with various modifications as are suited to the particular use contemplated. It is intended that the claims submitted herewith define the overall scope.

1-27. (canceled)

28. A surgical generator, comprising:
an ultrasonic energy circuit configured to generate an ultrasonic energy signal for activating an ultrasonic blade of a surgical instrument;
a radiofrequency (RF) energy circuit configured to generate an RF energy signal for activating an electrode of the surgical instrument; and
a control circuit configured to:
store a plurality of energy delivery algorithms, each of the energy delivery algorithms comprising instructions executable to cause the ultrasonic energy circuit and the RF energy circuit to generate the ultrasonic energy signal and the RF energy signal according to defined waveform parameters;
receive an algorithm selection signal from the surgical instrument, the algorithm selection signal designating a selected energy delivery algorithm from the plurality of energy delivery algorithms;
receive an activation signal from the surgical instrument; and
execute the selected energy delivery algorithm based on receiving the energy selection signal and the activation signal to automatically cause the ultrasonic energy circuit and the RF energy circuit to generate the ultrasonic energy signal and the RF energy signal according to the corresponding defined waveform parameters thereby activating the ultrasonic blade and the electrode.

29. The surgical generator of claim 28, wherein the plurality of energy delivery algorithms comprises a sequential energy delivery algorithm comprising instructions executable to automatically cause the ultrasonic energy circuit and the RF energy circuit to sequentially generate the ultrasonic energy signal and the RF energy signal.

30. The surgical generator of claim 28, wherein the plurality of energy delivery algorithms comprises a combined energy delivery algorithm comprising instructions executable to automatically cause the ultrasonic energy circuit and the RF energy circuit to concurrently generate the ultrasonic energy signal and the RF energy signal.

31. The surgical generator of claim 28, wherein the plurality of energy delivery algorithms comprises a duty cycle energy delivery algorithm comprising instructions executable to automatically cause the ultrasonic energy circuit and the RF energy circuit to cycle between generating the ultrasonic energy signal and the RF energy signal.

32. The surgical generator of claim 28, wherein the defined waveform parameters comprise a type of energy, a duty cycle, a voltage, a frequency, an impedance limit, a pulse width, or a current, or a combination thereof.

33. The surgical generator of claim 32, wherein the plurality of energy delivery algorithms comprises pre-defined algorithms and user-defined algorithms.

34. The surgical generator of claim 33, wherein the selected energy delivery algorithm is a pre-defined algorithm, and wherein the control circuit configured to:
receive a waveform parameter modification signal defining a modified waveform parameter for the selected energy delivery algorithm;
generate a user-defined algorithm from the pre-defined algorithm incorporating the modified waveform parameter.

35. The surgical generator of claim 28, wherein the ultrasonic circuit comprises a first terminal removably couplable to the surgical instrument, and wherein the RF energy circuit comprises a second terminal removably couplable to the surgical instrument.

36. The surgical generator of claim 28, further comprising a display, wherein the control circuit is configured to cause the display to indicate the selected energy delivery algorithm based on receiving the algorithm selection signal.

37. The surgical generator of claim 28, wherein the control circuit is configured to receive the plurality of energy delivery algorithms from a memory of the surgical instrument.

38. The surgical generator of claim 28, wherein the control circuit is configured to download the plurality of energy delivery algorithms from a remote device.

39. A system, comprising:
a surgical generator, comprising:
a first energy circuit configured to generate a first energy signal corresponding to a first type of energy;
a second energy circuit configured to a second energy signal corresponding to a second type of energy; and
a control circuit configured to:
store a plurality of energy delivery algorithms, each of the energy delivery algorithms comprising instructions executable to automatically cause the first energy circuit and the second energy circuit to generate the first energy signal and the second energy signal according to defined waveform parameters;
receive an algorithm selection signal corresponding to a selected energy delivery algorithm from the plurality of energy delivery algorithms;
receive an activation signal; and
execute the selected energy delivery algorithm based on receiving the energy selection signal and the activation signal to automatically cause the first energy circuit and the second energy circuit to generate the first energy signal and the second energy signal according to the corresponding defined waveform parameters; and
a surgical instrument couplable to the surgical generator, the surgical instrument comprising:
an end effector configured to:
deliver first energy for treating tissue based on the first energy signal generated by the first energy circuit; and
deliver second energy for treating tissue based on the second energy signal generated by the first energy circuit; and
a user interface configured to cause the surgical instrument to transmit the algorithm selection signal and the activation signal based on receiving a user input.

40. The system of claim 39, wherein the plurality of energy delivery algorithms comprises a duty cycle energy delivery algorithm comprising instructions executable to automatically cause the first energy circuit and the second energy circuit to cycle between generating the first energy signal and the second energy signal.

41. The system of claim 39, wherein the defined waveform parameters comprise a type of energy, a duty cycle, a voltage, a frequency, an impedance limit, a pulse width, or a current, or a combination thereof.

42. The system of claim 41, wherein the plurality of energy delivery algorithms comprises pre-defined algorithms and user-defined algorithms.

43. The system of claim 42, wherein the selected energy delivery algorithm is a pre-defined algorithm, and wherein the control circuit configured to:
receive a waveform parameter modification signal defining a modified waveform parameter for the selected energy delivery algorithm;
generate a user-defined algorithm from the pre-defined algorithm incorporating the modified waveform parameter.

44. The system of claim 39, wherein the first circuit comprises a first terminal removably couplable to the surgical instrument, and wherein the second energy circuit comprises a second terminal removably couplable to the surgical instrument.

45. The system of claim 39, further comprising a display, wherein the control circuit is configured to cause the display to indicate the selected energy delivery algorithm based on receiving the algorithm selection signal.

46. The system of claim 39, wherein the control circuit is configured to receive plurality of energy delivery algorithms from a memory of the surgical instrument.

47. The system of claim 39, wherein the first energy type is ultrasonic energy, wherein the second energy type is radiofrequency energy, and wherein end effector of the surgical instrument comprises:
an ultrasonic blade configured to deliver the ultrasonic energy based on the first energy signal generated by the first energy circuit;
an electrode configured to deliver the RF energy based on the second energy signal generated by the by the second energy circuit.
